SEQUENCE LISTING (30 sequences)

SEQ ID NO: 1 - RNA, 21 nt, synthetic construct
  source 1..21: mol_type = other RNA
acttctgattctcgttgccct

SEQ ID NO: 2 - RNA, 21 nt, synthetic construct
  source 1..21: mol_type = other RNA
caccctcgtgagcggagaact

SEQ ID NO: 3 - RNA, 22 nt, synthetic construct
  source 1..22: mol_type = other RNA
gtggccattcgacgacaggtta

SEQ ID NO: 4 - protein, 7 aa, synthetic construct
PKKKRKV

SEQ ID NO: 5 - protein, 16 aa, synthetic construct
KRPAATKKAGQAKKKK

SEQ ID NO: 6 - protein, 9 aa, synthetic construct
PAAKRVKLD

SEQ ID NO: 7 - protein, 11 aa, synthetic construct
RQRRNELKRSP

SEQ ID NO: 8 - protein, 38 aa, synthetic construct
NQSSNFGPMKGGNFGGRSSGPYGGGGQYFAKPRNQGGY

SEQ ID NO: 9 - protein, 42 aa, synthetic construct
RMRIZFKNKGKDTAELRRRRVEVSVELRKAKKDEQILKRRNV

SEQ ID NO: 10 - protein, 8 aa, synthetic construct
VSRKRPRP

SEQ ID NO: 11 - protein, 8 aa, synthetic construct
PPKKARED

SEQ ID NO: 12 - protein, 8 aa, synthetic construct
PQPKKKPL

SEQ ID NO: 13 - protein, 12 aa, synthetic construct
SALIKKKKKMAP

SEQ ID NO: 14 - protein, 5 aa, synthetic construct
DRLRR

SEQ ID NO: 15 - protein, 7 aa, synthetic construct
PKQKKRK

SEQ ID NO: 16 - protein, 10 aa, synthetic construct
RKLKKKIKKL

SEQ ID NO: 17 - protein, 10 aa, synthetic construct
REKKKFLKRR

SEQ ID NO: 18 - protein, 20 aa, synthetic construct
KRKGDEVDGVDEVAKKKSKK

SEQ ID NO: 19 - protein, 17 aa, synthetic construct
RKCLQAGMNLEARKTKK

SEQ ID NO: 20 - DNA, 7633 bp, synthetic construct
  source 1..7633: mol_type = other DNA
  misc_RNA 397..418: note = sgRNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggccattcgacgacaggttagttatagtactctggaaacagaatctactataacaaggcaaaatgccgtgtttatctcgtcaacttgttggcgagatttttttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatgtacccatacgatgttccagattacgctgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagcatatgactagtggcggtggcggagggatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccactagtggcggtggcggagggatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctagctagctttatttgtgaaatttgtgatgctattgctttatttgtaaccattataagctgcaataaacaagttaacaacaacaattgcattcattttatgtttcaggttcagggggagatgtgggaggttttttaaagcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 7393 bp, synthetic construct
  source 1..7393: mol_type = other DNA
  misc_RNA 397..418: note = sgRNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggccattcgacgacaggttagttatagtactctggaaacagaatctactataacaaggcaaaatgccgtgtttatctcgtcaacttgttggcgagatttttttagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgccaccatgtacccatacgatgttccagattacgctgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagcatatgactagtggcggtggcggagggatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccactagtggcggtggcggagggatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctagctagctttatttgtgaaatttgtgatgctattgctttatttgtaaccattataagctgcaataaacaagttaacaacaacaattgcattcattttatgtttcaggttcagggggagatgtgggaggttttttaaagcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 105 bp, synthetic construct
  misc_feature 1..22: note = RNA
  source 1..105: mol_type = other DNA
gtggccattcgacgacaggttagttatagtactctggaaacagaatctactataacaaggcaaaatgccgtgtttatctcgtcaacttgttggcgagattttttt

SEQ ID NO: 23 - DNA, 105 bp, synthetic construct
  misc_feature 1..22: note = RNA
  source 1..105: mol_type = other DNA
caccggtaagctgctgtccaatcaatatcatgagacctttgtcttagatgatattgttccgttttacggcacaaatagagcagttgaacaaccgctctaaaaaaa

SEQ ID NO: 24 - RNA, 21 nt, synthetic construct
  source 1..21: mol_type = other RNA
gaatggtcgtgggcaccggga

SEQ ID NO: 25 - RNA, 21 nt, synthetic construct
  source 1..21: mol_type = other RNA
acgctctcggaggggccgggc

SEQ ID NO: 26 - RNA, 21 nt, synthetic construct
  source 1..21: mol_type = other RNA
ggcaaacccgctaacctgtcg

SEQ ID NO: 27 - RNA, 21 nt, synthetic construct
  source 1..21: mol_type = other RNA
aagagagaggcggggaggagt

SEQ ID NO: 28 - RNA, 21 nt, Synthetic construct
  source 1..21: mol_type = other RNA
tggccattcgacgacaggtta

SEQ ID NO: 29 - DNA, 3438 bp, synthetic construct
  source 1..3438: mol_type = other DNA
cggacactagtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggcggttccggcggagggtccatgggcaaacggaactacatcctggggcttgccattgggataaccagcgttggctacggaattattgattatgagacacgcgatgtgattgacgccggggttaggctgttcaaagaggccaacgttgaaaacaacgagggaagacggagtaagcgcggagcaagaagactcaagcgcagacggagacatcggattcagagggtgaaaaagctgctcttcgattacaatctcctgaccgatcatagtgagctgagcggaatcaacccctacgaggcgcgagtgaaagggctttcccagaagctgtccgaagaggagttctccgccgcgttgctgcacctggccaaacggaggggggttcacaatgtaaacgaagtggaggaggacacgggcaatgaacttagtacgaaagaacagatcagtaggaactctaaggctctcgaagagaaatacgtcgctgagttgcagcttgagagactgaaaaaagacggcgaagtacgcggatctattaataggttcaagacttcagattacgtaaaggaagccaagcagctcctgaaagtacagaaagcgtaccatcagctcgatcagagcttcatcgatacctacatagatttgctggagacacggaggacatactacgagggcccaggggaaggatctccttttgggtggaaggacatcaaggaatggtacgagatgcttatgggacattgtacatattttccggaggagctcaggagcgtcaagtacgcctacaatgccgacctgtacaatgccctcaatgacctcaataacctcgtgattaccagggacgagaacgagaagctggagtactatgaaaagttccagattatcgagaatgtgtttaagcagaagaagaagccgacacttaagcagattgcaaaggaaatcctcgtgaatgaggaagatatcaagggatacagagtgacaagtacaggcaagcccgagttcacaaatctgaaggtgtaccacgatattaaggacataaccgcacgaaaggagataatcgaaaacgctgagctcctcgatcagatcgcaaaaattcttaccatctaccagtctagtgaggacattcaggaggaactgactaatctgaacagtgagctcacccaagaggaaattgagcagatttcaaacctgaaaggctacaccgggacgcacaatctgagcctcaaagcaatcaacctcattctggatgaactttggcacacaaatgacaaccaaattgccatattcaaccgcctgaaactggtgccaaaaaaagtggatctgtcacagcaaaaggaaatccctacaaccttggttgacgattttattctgtcccccgttgtcaagcggagcttcatccagtcaatcaaggtgatcaatgccatcattaaaaaatacggattgccaaacgatataattatcgagcttgcacgagagaagaactcaaaggacgcccagaagatgattaacgaaatgcagaagcgcaaccgccagacaaacgaacgcatagaggaaattataagaacaaccggcaaagagaatgccaagtatctgatcgagaaaatcaagctgcacgacatgcaagaaggcaagtgcctgtactctctggaagctatcccactcgaagatctgctgaataatccattcaattacgaggtggacgccatcatccctagatccgtaagctttgacaattccttcaataacaaagttctggttaaacaggaggaaaattctaaaaaagggaaccggaccccgttccagtacctgagctccagtgacagcaagattagctacgagacttttaagaaacatattctgaatctggccaaaggcaaaggcaggatcagcaagaccaagaaggagtacctcctcgaagaacgcgacattaacagatttagtgtgcagaaagatttcatcaaccgaaaccttgtcgatactcggtacgccacgagaggcctgatgaatctcctcaggagctacttccgcgtcaataatctggacgttaaagtcaagagcataaatgggggattcaccagctttctgaggagaaagtggaagtttaagaaggaacgaaacaaaggatacaagcaccatgctgaggatgctttgatcatcgctaacgcggactttatctttaaggaatggaaaaagctggataaggcaaagaaagtgatggaaaaccagatgttcgaggagaagcaggcagagtcaatgcctgagatcgagacagagcaggaatacaaggaaattttcatcacccctcatcagattaaacacataaaggacttcaaagactataaatactctcatagggtggacaaaaaacccaatcgcgagctcattaatgacaccctgtactcaacacggaaggatgataaaggtaataccttgattgtgaataatcttaatggattgtatgacaaagataacgacaagctcaagaagctgatcaacaagtctccagagaagctccttatgtatcaccacgacccacagacttatcagaaattgaaactgatcatggagcaatacggggatgagaagaacccactctacaaatattatgaggaaacaggtaattacctgaccaagtactccaagaaggataacggaccagtgatcaaaaagataaagtactatggcaacaaacttaatgcgcatttggacataactgacgattaccccaattctcgaaacaaggttgtgaagctctccctgaagccttatagatttgacgtgtacctggataatggggtttataaattcgtcaccgtgaaaaatctggacgtgatcaaaaaggagaactattatgaagtaaactcaaagtgctatgaggaggcgaagaagctgaagaagatctccaatcaggccgagttcatcgcttccttctataataacgatctcatcaagatcaatggagagctttatcgcgtcattggtgtgaacaatgacttgctgaacaggatcgaagtcaatatgatagacattacctaccgggagtatctcgaaaacatgaatgataaacggccgcctcgcatcatcaagacaatcgcatctaaaactcagtcaataaaaaagtactctaccgatatcctggggaatctctatgaagtgaagtcaaagaagcacccacaaatcattaaaaaaggtggatccgcagaagcagcagcaaaggaagcagcagcaaaggaagcagcagcaaaggcagtcgacaccggt

SEQ ID NO: 30 - DNA, 3249 bp, synthetic construct
  source 1..3249: mol_type = other DNA
ccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

(19) United States
(12) Patent Application Publication
(10) Pub. No.: US 2025/0177568 A1
(43) Pub. Date: Jun. 5, 2025

(54) COMPOSITIONS AND METHODS FOR MODULATING ALPHA-SYNUCLEIN EXPRESSION

(71) Applicants: Deniz Kirik, Lund (SE); The Board of Trustees of the Leland Stanford Junior University, Stanford, CA (US)

(72) Inventors: C. Alejandra Morato Torres, Stanford, CA (US); Faria Zafar, Stanford, CA (US); Lei S. Qi, Stanford, CA (US); Birgitt Schüle, Stanford, CA (US); Deniz Kirik, Lund (SE)

(73) Assignee: The Board of Trustees of the Leland Stanford Junior University, Stanford, CA (US)

(21) Appl. No.: 18/845,133

(22) PCT Filed: Mar. 16, 2023
(86) PCT No.: PCT/US2023/064587

§ 371 Date: Sep. 9, 2024

Related U.S. Application Data
(60) Provisional application No. 63/321,035, filed on Mar. 17, 2022.

(51) Int. Cl. A61K 48/00; A61K 9/00; A61P 25/16; C12N 9/22; C12N 15/11; C12N 15/86

(52) U.S. Cl. CPC A61K 48/005; A61K 9/0085; A61K 48/0075; A61P 25/16; C12N 9/22; C12N 15/111; C12N 15/86; C07K 2319/09; C07K 2319/81; C12N 2310/20; C12N 2750/14143

(57) ABSTRACT

This disclosure provides recombinant DNA mol-encoding fusion proteins that are able to regulate expression of alpha-synuclein. Also provided are various compositions comprising the recombinant DNA molecules, as well as associated methods of use. The recombinant DNA molecules and associated methods are useful for the treatment of subjects having disorders caused by excess expression or intracellular accumulation of alpha-synuclein, including Parkinson's disease.

### CROSS-REFERENCE TO RELATED APPLICATIONS

This application claims priority to and the benefit of U.S. Provisional Application No. 63/321,035, entitled “COMPOSITIONS AND METHODS FOR MODULATING ALPHA-SYNUCLEIN EXPRESSION” and filed on larch 17, 2022, the entire contents of which are herein incorporated by reference for all purposes.

### SEQUENCE LISTING

The official copy of the sequence listing is submitted electronically via USPTO Patent Center as an .st26 XML formatted sequence listing with a file named “S22-089_079445-009610PC-1370268_st26.xml”, created on Mar. 16, 2023, and having a size of 50 kb, and is filed concurrently with the specification herewith. The sequence listing contained in this XML formatted document is part of the specification and is herein incorporated by reference in its entirety.

### BACKGROUND

Parkinson's disease represents the second most common neurodegenerative disease of aging after Alzheimer's disease. Parkinson's disease prevalence rates are 1-2% of persons over age 65 and 4-5% for over age 85. Current treatments are entirely symptomatic, and to date, there are no available therapies proven to cure, halt, or slow disease progression. This represents a significant unmet medical need to develop innovative neuromnodulatory therapeutic approaches.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, provided herein are recombinant DNA molecules comprising a nucleic acid sequence that can encode a fusion protein comprising a CRISPR-associated nuclease and a transcriptional repressor. In some embodiments, the recombinant DNA molecules can further comprise a nucleic acid sequence that encodes a guide RNA that is complementary to a sequence in an alpha-synuclein gene or a gene regulatory region thereof. In some embodiments, the alpha-synuclein gene can be a human or non-human primate alpha-synuclein gene. In some embodiments, the nucleic acid sequence that encodes a guide RNA comprises a sequence that is at least 90%, 91%, 92%, 93%, 94%, 95%, 96%, 97%, 98%, or 99% (or greater) identical to SEQ ID NO:1, 2, or 3.

In some embodiments, the CRISPR associated nuclease of a fusion protein encoded by a recombinant DNA molecule provided herein is a Cas9. In some embodiments, the Cas9 can be Staphylococcus aureus (S. aureus) Cas9. In some embodiments, the CRISPR-associated nuclease can comprise one or more mutations relative to the wild-type CRISPR-associated nuclease. In some embodiments, at least one of the one or more mutations can result in a reduction in CRISPR-associated nuclease catalytic activity. In some embodiments, the one or more mutations can result in a catalytically inactive CRISPR-associated nuclease. In some embodiments, the CRISPR-associated nuclease can be S. aureus Cas9 and can comprise a D10A mutation and/or a N580A mutation.

In some embodiments, the transcriptional repressor of a fusion protein encoded by a recombinant DNA molecule provided herein can be or comprises a KRAB domain. In some embodiments, the fusion protein comprises at least 1 KRAB domain. In some embodiments, the fusion protein can comprise at least 2 KRAB domains. In some embodiments, the fusion protein can further comprise a nuclear localization signal.

Also provided are DNA constructs comprising any of the recombinant DNA molecules according to the present disclosure operably linked to a promoter. In some embodiments, the promoter can be a human MECP2 promoter, a human synapsin promoter, or a human PGK promoter. In some embodiments, the DNA construct can comprise two promoters: a first promoter operably linked to a nucleic acid sequence that can encode a fusion protein comprising a CRISPR-associated nuclease and a transcriptional repressor; and a second promoter operably linked to a nucleic acid sequence that can encode a guide RNA that is complementary to a sequence in an alpha-synuclein gene or a gene regulatory region thereof. In some embodiments, the first promoter can be a human MECP2 promoter, a human synapsin promoter, or a human PGK promoter. In some embodiments, the second promoter can be a human U6 promoter.

Also provided herein are are vectors comprising any of the DNA constructs provided herein. In some embodiments, the vector can be a viral vector. In some embodiments, the vector can be an adeno-associated virus vector. In some embodiments, the adeno-associated virus can be AAV9. In some embodiments, the vector can be up to 4800 bp in length. In some embodiments, the vector can be up to 5100 bp in length.

Also provided herein are isolated viruses comprising any of the vectors provided herein. In some embodiments, the virus can be an adeno-associated virus. In some embodiments, the adeno-associated virus serotype can be AAV9.

Also provided herein are compositions (e.g., pharmaceutical compositions) comprising (1) any of the recombinant DNA molecules, DNA constructs, vectors, or isolated viruses provided herein and (2) a pharmaceutically acceptable carrier.

Also provided herein are methods of treating a subject with Parkinson's disease. Methods as described herein can comprise administering to the subject (also referred to herein as the “subject in need” or “subject in need thereof”) a therapeutically effective amount (also referred to herein as an “effective amount”) of a composition as provided herein. In some embodiments, the composition can be administered intrathecally. In some embodiments, the composition can be administered into the cisterna magna. In some embodiments, the composition can be administered into cerebrospinal fluid. In some embodiments, administration of the composition can result in a decreased amount of alpha-synuclein protein expression in the subject relative to the amount of alpha-synuclein protein expression in the subject prior to administration of the composition.

Other objects, features, and advantages of the present disclosure will be apparent to one of skill in the art from the following detailed description and figures.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a part of the alpha synuclein (SNCA) genomic locus and sgRNAs chosen for screening, according to aspects of this disclosure. The sgRNAs that were found to down-regulate aSYN protein levels by more than 40% are indicated: sgRNA3, sgRNA4, and sgRNA6.

FIG. 2 shows the results of an sgRNA screen in HEK293 cells, according to aspects of this disclosure. Three sgRNAs, named sgRNA3, sgRNA4 and sgRNA6, down-regulate alpha-synuclein protein levels when expressed together with dSaCas9KRAB construct. Cells were harvested and collected 72 h after transfection (three wells/experiments and at least 6 experiments for each condition); their alpha-synuclein protein concentrations were measured using highly sensitive alphaLISA assay. aSYN levels were normalized to total protein levels. Statistical analysis was done using paired t-test, **p<0.005, ***p<0.001.

FIG. 3 shows the sequence of the sgRNA 6 sequence with optimized saCas9 scaffold, according to aspects of this disclosure. RNA6 is 100% homologeous to non-human primate sequence for Rhesus macaque (Macaca mulatta), Squirrel monkey (Saimiri), and Cynomolgus (Macaca fascicularis).

FIG. 4 shows a map of an expression cassette with hU6 promoter and RNA6, SYN1 or MECP2 promoter driving sadCas9 expression, flanked by ITR elements, according to aspects of this disclosure.

FIG. 5 shows that cisterna magna delivery of AAV9 virus vector allows efficient transduction throughout the mouse brain for both MECP2 and SYN1 promoter, according to aspects of this disclosure. The plots depict AAV9-mediated Cas9 expression throughout the brain. In all coronal section from olfactory bulb (OB), striatum (ST), midbrain or mesencephalon (MES), and cerebellum (CER), Cas9 mRNA fluorescent signal was detected as small pits (size 1-8 um) and large pits (size 8-16 um). Data are from three technical replicates (consecutive 15 um sections) per brain.

FIG. 6 shows that AAV9-mediated Cas9 expression facilitates significant alpha-synuclein downregulation via small guide RNA 6, according to aspects of this disclosure. The plots depict small guide RNA6 mediated downregulation of alpha-synuclein in different brain regions. In all coronal section from olfactory bulb (OB), striatum (ST), midbrain or mesencephalon (MES), and cerebellum (CER), SNCA mRNA fluorescent signal was detected as small pits (size 1-8 um) and large pits (size 8-16 um). Small guide RNA Gal4 is not targeting the human genome as serves as a negative control. Data are from three technical replicates (consecutive 15 um sections) per brain. ** is the p-value of <0.01.

FIGS. 7A-7B show expression of SadCas9 and alpha-synuclein expression, according to aspects of this disclosure. Shown are representative fluorescence images of the mouse brain tissue (olfactory bulb, striatum, mesencephalon, and cerebellum) for in situ hybriziation of SadCas9 (red) and alpha-synuclein (green) with nuclei (blue) for the two expression cassettes: MECP2 promoter-drive cassette (FIG. 7A: intrathecal, 1 month post-surgery; in situ hybridization RNAscope: Cas9 (red) and SNCA (green)) and SYN1 promoter-driven cassette (FIG. 7B: intrathecal, 1 month post-surgery: In situ hybridization RNAscope: Cas9 (red) and SNCA (green)) compared between RNA6 and control guide RNA. Quantitative data from these images are presented in FIG. 5 and FIG. 6.

FIGS. 8A-8F illustrate various aspects of screening of sgRNAs targeting transcriptional start site 2 in the human SNCA promoter (TSS2) in the alpha synuclein (SNCA) promoter with HEK293 cells. (FIG. 8A) Schematic representation of vector cassettes for inducible SadCas9 and red fluorescent protein (RFP) and sgRNAs and blue fluorescent protein (BFP) co-expression. (FIG. 8B) UCSC Genome browser view of SNCA promoter region showing five sgRNAs targeted to the transcriptional start site 2 in the human SNCA promoter. (FIG. 8C) Schematic overview of the transient inducible expression of SadCas9 and sgRNA, fluorescent activated cell sorting (FACS) and quantitative PCR amplification with Taqman probes. (FIG. 8D) Representative images of induced expression of SadCas9 with different sgRNAs and negative SadCas9 only control. (FIG. 8E) FACS sorting and gating of RFP and BFP double-positive cells, on average we reached 53.24% double positive cells in HEK293 cultures. (FIG. 8F) Taqman™ quantitative expression analysis of the SNCA gene, normalized to GAPDH, and compared to SadCas9 only cells (this % number (53.38%) relates to FIG. 8E with the double positive cells in the upper right panel).

FIG. 9 is an illustration of an embodiment of study design and an embodiment of a timeline of experiments according to the present disclosure. Four-month-old Dbl-PAC-Tg (SNCA^{A53T}) mice were grouped into six experimental Groups: Groups 1 and 4 received AAV9 virus containing the active construct of SadCas9 with TSS2 sgRNA2 stereotactically injected into the substantia nigra: Groups 2 and 5 received AAV9 containing a non-human targeting sgRNA gal4 control; and Groups 3 and 6 received saline controls. Groups 1-3 were euthanized after 1 month and groups 4-6 after 6 months and brains and peripheral organs were harvested and fresh frozen in liquid nitrogen. Coronal sections of 20 um were taken and further stained by in situ hybridization RNAScope or immunofluorescence histology, quantified, and analyzed by Qpath (v 0.4.2).

FIGS. 10A-10L show aspects of SNCA mRNA downregulation at 1-month post-surgery according to an embodiment of the present disclosure. SNCA mRNA shows a reduction illustrated as RNAScope foci per cell in the TSS2-sg2 group (FIGS. 10I1 and I2: FIG. 10L) for the substantia nigra. SNCA mRNA signals are unchanged in the hippocampal (FIGS. H1-10H2; FIG. 10K) and cortical areas (FIGS. 10G1-10G2; FIG. 10J). RNA expression was detected using specific in situ hybridization probes (RNAScope) against alpha-synuclein (green) and Cas9 (red), counterstained with DAPI (blue). Panoramic coronal mesencephalic section of brains one-month post-surgery are shown as saline control (top left panel), control (ctrl) sgRNA (top middle panel), and active TSS2-sgRNA2 (top right panel) (scale bar=500 um). In-situ hybridization signal merged images depict SNCA mRNA (aSyn, green), sadCas9 (red), and DAPI (blue). Respective regions of cortex, dentate gyrus of hippocampus, and substantia nigra at higher magnification of the injected side are shown for saline control group (left panel figures starting one down from the top, FIGS. 10A 1/A2, B1/B2, C1/C2), ctrl sgRNA group (middle panel figures starting one down from the top, FIGS. 10D1/D2, E1/E2, F1/F2), and TSS2-sg2 group (right panel figures starting one down from the top, FIGS. 10G1/G2, H1/H2, I1/I2). Scale bars are indicated at 50 μm (FIGS. 10A1, B1, C1, D1, E1, F1, H1, and I1) and 15 μm (FIGS. 10A2, B2, C2, D2, E2, F2, G2, H2, I2). FIGS. 10K-L are bar graphs quantifying the SNCA mRNA expression signal between the three treatment groups (saline, control sgRNA, TSS2-sg2) according to an embodiment of the present disclosure. Results are shown as ns=non-statistically significant. (*) p<0.05, (**) p<0.01, or (***) p<0.001.

FIGS. 11A-11L show aspects of SadCas9-mediated alpha-synuclein protein downregulation at 1 month post stereotactic surgery in the substantia nigra according to an embodiment of the present disclosure. Alpha-synuclein immunostaining shows a reduction of signal intensity in the TSS2-sg2 group (right panel) for the substantia nigra and hippocampus (FIGS. 11G1-G2, H1-I1; FIG. 11K, FIG. 11L), whereas alpha-synuclein signals are unchanged in the cortical area (FIGS. 11G1-G2; FIG. 11J). Panoramic coronal mesencephalic section of brains one-month post-surgery are shown as saline control (top left panel), control (ctrl) sgRNA (top middle panel), and active TSS2-sgRNA2 (top right panel) (scale bar=500 μm). Immunofluorescence merged images depict alpha-synuclein (aSyn, green), sadCas9 (red), and DAPI (blue). Respective regions of cortex, dentate gyrus of hippocampus, and substantia nigra at higher magnification of the injected side are shown for saline control group (left panel figures starting one down from the top. FIGS. 11A1/A2, B1/B2, C1/C2), ctrl sgRNA group (middle panel figures starting one down from the top. FIGS. 11D1/D2, E1/E2, F1/F2), and TSS2-sg2 group (right panel figures starting one down from the top, FIGS. 11G1/G2, H1/H2, I1/I2). Scale bars are indicated at 50 μm (FIGS. 11A1, B1, C1, D1, E1, F1, H1, and I1) and 15 μm (FIGS. 11A2, B2, C2, D2, E2, F2, G2, H2, I2). FIGS. 11K-L depicts bar graphs quantifying the a-syn expression signal between the three treatment groups (saline, control sgRNA, TSS2-sg2) according to an embodiment of the present disclosure. Results are shown as ns=non-statistically significant, (*) p<0.05, (**) p<0.01, or (***) p<0.001.

FIGS. 12A-12L show aspects of microglia activation at 1 month post stereotactic surgery in the substantia nigra according to an embodiment of the present disclosure. Ibal immunoreactivity is increased in TSS2-sg2 and ctrl sgRNA group (right and middle panels, FIGS. 12F1-F2, FIGS. 12I1-I2; FIG. 12L) for the substantia nigra, but not in the hippocampus or cortical region (FIGS. 12E1-E2, FIGS. 12D1-12D2; FIG. 12K). Panoramic coronal mesencephalic section of brains one-month post-surgery are shown as saline control (top left panel), control (ctrl) sgRNA (top middle panel), and active TSS2-sgRNA2 (top right panel) (scale bar=500 μm). Immunofluorescence merged images depict sadCas9 (red), Ibal (white), Cd16/32 (green) and DAPI (blue). Respective regions of cortex, dentate gyrus of hippocampus, and substantia nigra at higher magnification of the injected side are shown for saline control group (left panel figures starting one down from the top, FIGS. 12A1/A2, B1/B2, C1/C2), ctrl sgRNA group (middle panel figures starting one down from the top, FIGS. 12D1/D2, E1/E2, F1/F2), and TSS2-sg2 group (right panel figures starting one down from the top, FIGS. 12G1/G2, H1/H2, I1/I2). Scale bars are indicated at 50 μm (FIGS. 12A1, B1, C1, D1, E1, F1, H1, and I1) and 15 μm (FIGS. 12A2, B2, C2, D2, E2, F2, G2, H2, I2). FIGS. 12K-L depicts bar graphs quantifying the % Ibal microglia signal between the three treatment groups (saline, control sgRNA, TSS2-sg2) according to an embodiment of the present disclosure. Results are shown as ns=non-statistically significant, (*) p<0.05, (**) p<0.01, or (***) p<0.001.

FIGS. 13A-13L show aspects of SNCA mRNA downregulation at 6-months post-surgery according to an embodiment of the present disclosure. SNCA mRNA shows a reduction illustrated as RNAScope foci per cell in the TSS2-sg2 group (top right panel) for the substantia nigra and cortex. SNCA mRNA signals are unchanged in the hippocampal area. RNA expression is detected using specific in situ hybridization probes (RNAScope) against alpha-synuclein (green) and Cas9 (red) counterstained with DAPI (blue). Panoramic coronal mesencephalic section of brains 6 months post-surgery are shown as saline control (top left panel), control (ctrl) sgRNA (top middle panel), and active TSS2-sgRNA2 (top right panel) (scale bar=500 μm). In situ hybridization signal merged images depict SNCA mRNA (aSyn, green), sadCas9 (red), and DAPI (blue). Respective regions of cortex, dentate gyrus of hippocampus, and substantia nigra at higher magnification of the injected side are shown for saline control group (left panel figures starting one down from the top, FIGS. 13A1/A2, B1/B2, C1/C2), ctrl sgRNA group (middle panel figures starting one down from the top, FIGS. 13D1/D2, E1/E2, F1/F2), and TSS2-sg2 group (right panel figures starting one down from the top, FIGS. 13G1/G2, H1/H2, I1/I2). Scale bars are indicated at 50 μm (FIGS. 13A1, B1, C1, D1, E1, F1, H1, and I1) and 15 μm (FIGS. 13A2, B2, C2, D2, E2, F2, G2, H2, I2). FIGS. 13K-L depicts bar graphs quantifying the SNCA mRNA expression signal between the three treatment groups (saline, control sgRNA, TSS2-sg2) according to an embodiment of the present disclosure. Results are shown as ns=non-[statistically] significant, (*) p<0.05, (**) p<0.01, or (***) p<0.001.

FIGS. 14A-14L show aspects of SadCas9-mediated alpha-synuclein protein downregulation 6 months post stereotactic surgery in substantia nigra and hippocampus according to an embodiment of the present disclosure. Alpha-synuclein immunostaining shows a reduction of signal intensity in the TSS2-sg2 group for the substantia nigra and hippocampus (FIGS. 14I-I2, FIGS. 14H1-H2, respectively), whereas alpha-synuclein signals are unchanged in the cortical area (FIGS. 14G1-G2). Panoramic coronal mesencephalic section of brains 6 months post-surgery are shown as saline control (top left panel), control (ctrl) sgRNA (top middle panel), and active TSS2-sgRNA2 (top right panel) (scale bar=500 μm). Immunofluorescence merged images depict alpha-synuclein (aSyn, green), sadCas9 (red), and DAPI (blue). Respective regions of cortex, dentate gyrus of hippocampus, and substantia nigra at higher magnification of the injected side are shown for saline control group (left panel figures starting one down from the top, FIGS. 14A1/A2, B1/B2, C1/C2), ctrl sgRNA group (middle panel figures starting one down from the top, FIGS. 14D1/D2, E1/E2, F1/F2), and TSS2-sg2 group (right panel figures starting one down from the top, FIGS. 14G1/G2, H1/H2, I1/I2). Scale bars are indicated at 50 μm (FIGS. 14A1, B1, C1, D1, E1, F1, H1, and I1) and 15 μm (FIGS. 14A2, B2, C2, D2, E2, F2, G2, H2, I2). FIGS. 14K-L depicts bar graphs quantifying the a-syn expression signal between the three treatment groups (saline, control sgRNA, TSS2-sg2) according to an embodiment of the present disclosure. Results are shown as ns=non-[statistically] significant, (*) p<0.05, (**) p<0.01, or (***) p<0.001.

FIGS. 15A-15L show aspects of microglial activation 6-months post stereotactic surgery in the substantia nigra according to an embodiment of the present disclosure. Ibal immunoreactivity is unchanged between the three treatment groups (saline, control sgRNA, TSS2-sg2). Panoramic coronal mesencephalic section of brains 6 months post-surgery are shown as saline control (top left panel), control (ctrl) sgRNA (top middle panel), and active TSS2-sgRNA2 (top right panel) (scale bar=500 μm). Immunofluorescence merged images depict sadCas9 (red), Ibal (white). Cd16/32 (green) and DAPI (blue). Respective regions of cortex, dentate gyrus of hippocampus, and substantia nigra at higher magnification of the injected side are shown for saline control group (left panel figures starting one down from the top, FIGS. 15A1/A2, B1/B2, C1/C2), ctrl sgRNA group (middle panel figures starting one down from the top, FIGS. 15D1/D2, E1/E2, F1/F2), and TSS2-sg2 group (right panel figures starting one down from the top. FIGS. 15G1/G2, H1/H2, I1/I2). Scale bars are indicated at 50 μm (FIGS. 15A1, B1, C1, D1, E1, F1, H1, and I1) and 15 μm (FIGS. 15A2, B2, C2, D2, E2, F2, G2, H2, I2). FIGS. 15K-L depicts bar graphs quantifying the % Ibal microglia signal between the three treatment groups (saline, control sgRNA, TSS2-sg2) according to an embodiment of the present disclosure. Results are shown as ns=non-[statistically] significant. (*) p<0.05, (**) p<0.01, or (***) p<0.001.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art: therefore, information well known to the skilled artisan is not necessarily included.

### I. Terminology

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e, at least one) of the grammatical object of the article. By way of example, “an element” means at least one element and can include more than one element.

The use herein of the terms “including.” “comprising,” or “having,” and variations thereof, is meant to encompass the elements listed thereafter and equivalents thereof as well as additional elements. Embodiments recited as “including,” “comprising.” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the combination of features as recited in the claims. See, In re Herz. 537 F.2d 549, 551-52, 190 U.S.P.Q. 461, 463 (CCPA 1976) (emphasis in the original): see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X. 0.97X, 0.98X. 0.99X, 1.01X, 1.02X. 1.03X. 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the present disclosure also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof, alleles, orthologs, SNPs, and complementary sequences as well as the sequence explicitly indicated.

The term “identity” or “substantial identity.” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein: preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch. J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff. Proc. Natl. Acad Sci. USA 89:10915 (1989)).

The BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

Unless otherwise indicated, a particular nucleic acid sequence can also implicitly encompass conservatively modified variants thereof, alleles, orthologs, SNPs, and complementary sequences as well as the sequence explicitly indicated.

The terms “transfection”, “transduction”, “transfecting” or “transducing” can be used interchangeably and are defined as a process of introducing a nucleic acid molecule or a protein to a cell. Nucleic acids can be introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” can also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements can be encoded by the same plasmid. Expression in trans refers to the instance where the gene and the regulatory elements can be encoded by separate plasmids.

The term “vector” refers to a carrier nucleic acid molecule into which a nucleic acid sequence can be inserted for introduction into a host cell. In some embodiments, vectors of use in the present disclosure are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector, e.g., a promoter. Vectors include non-viral vectors such as plasmids and viral vectors.

A “viral vector” is a viral-derived nucleic acid that is capable of transporting another nucleic acid into a cell (which also can be packaged in a virus that can subsequently infect a cell). A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “operably linked” refers to a functional linkage between a first nucleic acid sequence and a second nucleic acid sequence, such that the first sequence is transcribed (if the operable linkage is to a promoter) or such that the first and second nucleic acid sequences are transcribed into a single nucleic acid sequence. Operably linked nucleic acid sequences need not be physically adjacent to each other. The term “operably linked” also refers to a functional linkage between a nucleic acid expression control sequence (such as a promoter, or array of transcription factor binding sites) and a transcribable nucleic acid sequence, wherein the expression control sequence directs transcription of the nucleic acid corresponding to the transcribable sequence.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

An “antisense” oligonucleotide or polynucleotide is a nucleotide sequence that is substantially complementary to a target polynucleotide or a portion thereof and has the ability to specifically hybridize to the target polynucleotide.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The amino acids in the polypeptides described herein can be any of the 20 naturally occurring amino acids. D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-587 (2013); Xie et la. “Adding amino acids to the genetic repertoire,” 9 (6): 548-54 (2005)); and all references cited therein. Beta and gamma amino acids are known in the art and are also contemplated herein as unnatural amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A). Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

As used herein, terms as used herein have the meanings ascribed to them unless specified otherwise. Other terms used in the fields of recombinant nucleic acid technology, microbiology, immunology, antibody engineering, and molecular and cell biology as used herein will be generally understood by one of ordinary skill in the applicable arts.

### II. Introduction

The present disclosure is based in part on the discovery by the inventors of a novel gene engineering strategy that utilizes CRISPR interference technology and facilitates mRNA transcript reduction without introducing permanent mutations at the genomic level. As demonstrated in the Examples herein, the strategy was used to target the promoter region of the alpha-synuclein gene, which has been implicated in the pathophysiology of Parkinson's disease. There is strong evidence that that an excess or aggregation of alpha-synuclein protein (Genc ID: 6622) can lead to neurodegeneration and Parkinson's disease. Clinical genetic studies of families with autosomal-dominant parkinsonism show that gene copy number or point mutations of the alpha-synuclein genomic locus are causative for disease. There is an apparent gene-dosage effect clinically documented in these families with an earlier age at onset, more severe clinical symptoms, and faster disease progression in individuals with an alpha-synuclein genomic triplication than alpha-synuclein genomic duplication or sporadic Parkinson's disease. Additionally, increased expression of alpha-synuclein is known to facilitate protein aggregation and neurodegeneration. Without being bound by any particular theory, it is thought that he mechanism of action for the CRISPR gene-engineering strategy is based on the principle that a small guide RNA, complementary to a target sequence, guides CRISPR/dCas9 mutant protein to the promoter region of the alpha-synuclein gene (Gene ID: 6622), and that this binding sterically hinders or modulates gene transcription, resulting in reduced mRNA expression and reduced translated alpha-synuclein protein.

The unique strategy described herein of manipulating alpha-synuclein gene expression at the DNA level prevents alpha-synuclein from being produced, rather than eliminating the gene/protein product. It alleviates impairment at all cellular components usually affected by alpha-synuclein-mediated toxicity. This approach meets a significant unmet medical need by altering the disease course of Parkinson's disease and/or significantly improving the treatment of symptoms beyond current standards of care.

### III. Recombinant DNA Molecules

In one aspect, provided herein are recombinant DNA molecules comprising a nucleic acid sequence that encodes a fusion protein comprising a CRISPR-associated nuclease and a transcriptional regulatory domain. In some embodiments, the transcriptional regulatory domain is a transcriptional repressor. In some embodiments, the fusion proteins encoded by the recombinant DNA molecules provided herein are useful for modulating (e.g., inhibiting or decreasing) transcription of a target gene. In some embodiments, the recombinant DNA molecules provided herein further comprise a nucleic acid sequence that encodes one or more guide polynucleotides (e.g., guide RNAs).

### A. CRISPR-Associated Nuclease

Any suitable CRISPR-associated nuclease can be used in the fusion proteins disclosed herein. In some embodiments, the CRISPR-associated (Cas) nuclease is derived from a naturally occurring CRISPR (Clustered Regularly Interspaced Short Palindromic Repeats)/Cas (CRISPR-associated) system. In bacteria, this system can provide adaptive immunity against foreign DNA (Barrangou. R., et al. “CRISPR provides acquired resistance against viruses in prokaryotes, “Science (2007) 315:1709-1712; Makarova. K. S., et al. “Evolution and classification of the CRISPR-Cas systems,” Nat Rev Microbiol (2011) 9:467-477: Garneau, J. E., et al. “The CRISPR/Cas bacterial immune system cleaves bacteriophage and plasmid DNA,” Nature (2010) 468:67-71: Sapranauskas, R., et al. “The Streptococcus thermophilus CRISPR/Cas system provides immunity in Escherichia coli,” Nucleic Acids Res (2011) 39:9275-9282). Suitable Cas nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides, or any functional fragment thereof, any derivative thereof; any variant thereof; and any fragment thereof.

In a wide variety of organisms including diverse mammals, animals, plants, microbes, and yeast, a CRISPR/Cas system (e.g., modified and/or unmodified) can be utilized as a genome engineering tool. A CRISPR/Cas system can comprise a guide nucleic acid such as a guide RNA (gRNA: described further below) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al. “Cas9-crRNA ribonucleoprotein complex mediates specific DNA cleavage for adaptive immunity in bacteria,” Proc Natl Acad Sci USA (2012) 109: E2579-E2 86: Jinek, M., et al, “A programmable dual-RNA-guided DNA endonuclease in adaptive bacterial immunity,” Science (2012) 337:816-821; Sternberg, S. H., et al. “DNA interrogation by the CRISPR RNA-guided endonuclease Cas9.” Nature (2014) 507:62: Deltcheva, E., et al, “CRISPR RNA maturation by trans-encoded small RNA and host factor RNase III,” Nature (201 1) 471:602-607), and has been widely used for programmable genome editing in a variety of organisms and model systems (Cong, L. et al, “Multiplex genome engineering using CRISPR Cas systems.” Science (2013) 339:819-823: Jiang. W. et al, “RNA-guided editing of bacterial genomes using CRISPR-Cas systems,” Nat. Biotechnol. (2013) 31:233-239. Sander, J. D. & Joung, J. K. “CRISPR-Cas systems for editing, regulating and targeting genomes,” Nature Biotechnol. (2014) 32:347-355).

Any suitable CRISPR/Cas system can be used. A CRISPR/Cas system can be referred to using a variety of naming systems. Exemplary naming systems are provided in Makarova. K. S. et al. “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1. Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

A nuclease comprising a Cas protein can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH). DNA binding domain. RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

Non-limiting examples of Cas proteins include c2c1. C2c2, c2c3, Cas1, Cas1B, Cas2, Cas3, Cas4, Cas5, Cas5c (CasD), Cash, Cashe, Cas6f, Cas7, Cas8a, Cas8a1, Cas8a2, Cas8b. Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17. Csx14, Csx10, Csx16, CsaX, Csx3, Csx1, Csx15, Csf1, Csf2, Csf3, Csf4, and Cu1966, and homologs or modified versions thereof. In some embodiments, the CRISPR-associated nuclease of the fusion protein described herein is Cas9.

A Cas protein can be from any suitable organism. Non-limiting examples include Streptococcus pyogenes. Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas nap hthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicendosiruptor becscit, Candidatus Desulforuchs, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africames, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia mucinphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans. Rhodospirillum rubrum. Candidates Puniceispirillum marinum, Verminephrobacter eiseniae, Ralstonia syzygii, Dinoroseobacter shibae, Azospirillum, Nitrobacter hamburgensis, Bradyrhizobium. Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutierella excrementihominis, Wolinella succinogenes, and Francisella novicida.

A Cas protein can comprise one or more nuclease domains, such as DNase domains. For example, a Cas9 protein can comprise a RuvC-like nuclease domain and/or an HNH-like 20 nuclease domain. The RuvC and HNH domains can each cut a different strand of double-stranded DNA to make a double-stranded break in the DNA. A Cas protein can comprise only one nuclease domain (e.g., Cpf1 comprises RuvC domain but lacks HNH domain). A Cas protein can comprise an amino acid sequence having at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a nuclease domain (e.g., RuvC domain, HNH domain) of a wild-type Cas protein.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking cleavage activity is an embodiment according to the present disclosure. In some embodiments, the Cas9 protein is a Cas9 protein from S. pyogenes (e.g., SwissProt accession number Q99ZW2). In some embodiments, the Cas9 protein is a Cas9 from S. aureus (e.g., SwissProt accession number J7RUA5). In some embodiments, the Cas9 protein is a modified version of a Cas9 protein from S. pyogenes or S. aureus. In some embodiments, the Cas9 protein is derived from a Cas9 protein from S. pyogenes or S. aureus. For example, a S. pyogenes or S. Aureus Cas9 protein lacking cleavage activity.

Cas9 can generally refer to a polypeptide with at least about 5%, 10%, 20%, 30%. 40%, 50%, 60%, 70%, 80%, 90%, 91%, 92%, 93%, 94%, 95%, 96%, 97%, 98%, 99% or 100% sequence identity and/or sequence similarity to a wild-type exemplary Cas9 polypeptide (e.g., Cas9 from S. aureus), Cas9 can refer to a polypeptide with at most about 5%, 10%, 20%, 30%, 40%, 50%, 60%, 70%, 80%, 90%, 91%, 92%, 93%, 94%, 95%, 96%, 97%, 98%, 99% or 100% sequence identity and/or sequence similarity to a wild-type exemplary Cas9 polypeptide (e.g., from S. aureus), Cas9 can refer to the wildtype or a modified form of the Cas9 protein that can comprise an amino acid change such as a deletion, insertion, substitution, variant, mutation, fusion, chimera, or any combination thereof.

A Cas protein as used herein can be a wild-type or a modified form of a Cas protein. A Cas protein can be an active variant, inactive variant, or fragment of a wild-type or modified Cas protein. In some embodiments, the fusion proteins encoded by the recombinant DNA molecules provided herein comprise a CRISPR-associated nuclease that comprises one or more mutations and/or modifications (e.g., a deletion, insertion, substitution, variant, mutation, fusion, chimera, or any combination thereof relative to a wild-type version of the Cas protein). A CRISPR-associated nuclease can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild-type exemplary CRISPR-associated nuclease. Other examples include codon-optimized Cas9 variants, and well as Cas9 fusion proteins that include a nuclear localization sequence (NLS), for example, a nucleoplasmin NLS signal.

In some embodiments, at least one of the one or more mutations and/or modifications results in a nuclease deficient protein or a protein with decreased nuclease activity relative to a wild-type Cas protein. A nuclease deficient protein can retain the ability to bind DNA, but may lack or have reduced nucleic acid cleavage activity. For example, the mutated and/or modified form of the Cas protein can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity (or any intervening range therein) of the wild-type Cas protein (e.g., Cas9 from S. aureus). A Cas nuclease (e.g., retaining wild-type nuclease activity, having reduced nuclease activity, and/or lacking nuclease activity) can function in a CRISPR/Cas system to regulate the level and/or activity of a target gene or protein (e.g., decrease, increase, or elimination). The Cas protein can bind to a target polynucleotide and prevent transcription by physical obstruction or edit a nucleic acid sequence to yield non-functional gene products. The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. In some embodiments, double strand break targeting specificity is improved by targeting a nickase to opposite strands at two nearby loci. If a nickase cleaves the single strand at both loci, a double strand break is formed and can be repaired via HR as described herein. If all of the nuclease domains of a Cas protein (e.g., both RuvC and HNH nuclease domains in a Cas9 protein: RuvC nuclease domain in a Cpf1 protein) are deleted or mutated, the resulting Cas protein can have a reduced or no ability to cleave both strands of a double-stranded DNA.

An example of a mutation that can convert a Cas9 protein into a dCas9 protein is a D10A (aspartate to alanine at position 10 of Cas9) mutation Cas9 from S. aureus. An additional example of a mutation that can convert a Cas9 protein into a dCas9 protein is a N580A (asparagine to alanine at amino acid position 580) in S. aureus Cas9. In some embodiments, the recombinant DNA molecules provided herein encode fusion proteins comprising Cas9 polypeptides comprising one or both of the D10A mutation and the N580A mutation.

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

### B. Transcription Regulatory Domains

In some embodiments, the fusion proteins encoded by the recombinant DNA molecules provided herein comprise one or more transcription regulatory domains (e.g., repressor domains, activator domains, epigenetic domains, recombinase domains, transposase domains, flippase domains, nickase domains, or any combination thereof). Non-limiting exemplary repression domains include the KRAB (Kruppel-associated box) domain of Koxl, the Mad mSIN3 interaction domain (SID), ERF repressor domain (ERD), and are described in Chavez et al., Nat Methods, 2015, 12 (4): 326-328 and U.S. Patent App. Publ. No. 20140068797. The one or more transcription regulatory domains can be fused to the CRISPR-associated nuclease in any suitable order and/or orientation. In some embodiments, the recombinant DNA molecules provided herein comprise a nucleic acid sequence encoding a fusion protein comprising at least one or at least two KRAB domains.

A fusion protein encoded by the recombinant DNA molecules provided herein may also comprise additional peptide sequences which are not involved in regulating gene expression, for example linker sequences, targeting sequences, etc. The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a CRISPR-associated nuclease) to a nucleus utilizing a nuclear localization signal (NLS): outside of a nucleus of a cell, for example to the cytoplasm, utilizing a nuclear export signal (NES); mitochondria utilizing a mitochondrial targeting signal: the endoplasmic reticulum (ER) utilizing an ER-retention signal: a peroxisome utilizing a peroxisomal targeting signal; plasma membrane utilizing a membrane localization signal: or combinations thereof.

In a preferred embodiment, a fusion protein as described herein comprises an NLS. Non-limiting examples of NLSs include an NLS sequence derived from: the NLS of the SV40 virus large T-antigen, having the amino acid sequence PKKKRKV (SEQ ID NO: 4); the NLS from nucleoplasmin (e.g, the nucleoplasmin bipartite NLS with the sequence KRPAATKKAGQAKKKK (SEQ ID NO: 5)): the c-myc NLS having the amino acid sequence PAAKRVKLD (SEQ ID NO: 6) or RQRRNELKRSP (SEQ ID NO: 7); the hRNPAI M9 NLS having the sequence NQSSNFGPMKGGNFGGRSSGPYGGGGQYFAKPRNQGGY (SEQ ID NO: 8): the sequence RMRIZFKNKGKDTAELRRRRVEVSVELRKAKKDEQILKRRNV (SEQ ID NO: 9) of the IBB domain from importin-alpha: the sequences VSRKRPRP (SEQ ID NO: 10) and PPKKARED (SEQ ID NO: 11) of the myoma T protein: the sequence PQPKKKPL (SEQ ID NO: 12) of human p53; the sequence SALIKKKKKMAP (SEQ ID NO: 13) of mouse c-abl IV: the sequences DRLRR (SEQ ID NO: 14) and PKQKKRK (SEQ ID NO: 15) of the influenza virus NSI: the sequence RKLKKKIKKL (SEQ ID NO: 16) of the Hepatitis virus delta antigen: the sequence REKKKFLKRR (SEQ ID NO: 17) of the mouse Mx1 protein: the sequence KRKGDEVDGVDEVAKKKSKK (SEQ ID NO: 18) of the human poly (ADP-ribose) polymerase; and the sequence RKCLQAGMNLEARKTKK (SEQ ID NO: 19) of the steroid hormone receptors (human) glucocorticoid.

A fusion protein as described herein can also comprise a heterologous polypeptide providing increased or decreased stability.

A fusion protein as described herein can also comprise a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AccGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFPI, DsRed-Express. DsRed2, DsRed-Monomer, HcRed-Tandem.

HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly (NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, SI, T7, V5, VSV-G, histidine (His), biotin carboxyl carrier protein (BCCP), and calmodulin.

In some embodiments, the various domains of the fusion proteins described herein are linked via a linker. A linker can be any linker known in the art.

### C. Guide Nucleic Acids

In some embodiments, a fusion protein encoded by a recombinant DNA molecule described herein is able to bind a guide nucleic acid (e.g., a guide RNA) that is complementary to a sequence in a target gene. In some embodiments, the recombinant DNA molecules provided herein comprise both a nucleic acid sequence encoding a fusion protein as described above and a nucleic acid sequence encoding one or more guide nucleic acids. In some embodiments, the guide nucleic acid and the fusion protein are encoded by separate recombinant DNA molecules. In some embodiments, a guide nucleic acid is ribonucleic acid (guide RNA).

In some embodiments, the CRISPR-associated nuclease of the fusion protein can be complexed with the at least one guide RNA polynucleotide. The at least one guide RNA polynucleotide can comprise a nucleic-acid targeting region that comprises a complementary sequence to a nucleic acid sequence on the targeted polynucleotide such as the targeted mammalian genomic loci, mammalian genes, human genomic loci, or human genes to confer sequence specificity of nuclease targeting. In some embodiments, the at least one guide RNA polynucleotide can comprise two separate nucleic acid molecules, which can be referred to as a double guide nucleic acid or a single nucleic acid molecule, which can be referred to as a single guide nucleic acid (e.g., single guide RNA or sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracrRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like molecule and a tracrRNA-like molecule.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

In some embodiments, the nucleic acid-targeting region of a guide nucleic acid can be between 18 to 72 nucleotides in length. The nucleic acid-targeting region of a guide nucleic acid (e.g., spacer region) can have a length of from about 12 nucleotides to about 100 nucleotides. For example, the nucleic acid-targeting region of a guide nucleic acid (e.g., spacer region) can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment can have a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The length of the nucleic acid-targeting region can be at least 5, 10, 15, 16, 17, 18, 19, 20, 21, 22, 23, 24, 25, 30 or more nucleotides. The length of the nucleic acid-targeting region (e.g., spacer sequence) can be at most 5, 10, 15, 16, 17, 18, 19, 20, 21, 22, 23, 24, 25, 30 or more nucleotides.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

A protospacer sequence of a targeted polynucleotide can be identified by identifying a protospacer-adjacent motif (PAM) within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility. % GC, frequency of genomic occurrence, methylation status, presence of SNPs, and the like.

The percent complementarity between the nucleic acid-targeting sequence (e.g., a spacer sequence of the at least one guide polynucleotide as disclosed herein) and the target nucleic acid (e.g., a protospacer sequence of the one or more target genes as disclosed herein) can be at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% over about 20 contiguous nucleotides.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

The Cas protein binding segment of a guide nucleic acid can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas protein-binding segment of a guide nucleic acid can have a length of from about 15 nucleotides (nt) to about 80 nt, from about 15 nt to about 50 nt, from about 15 nt to about 40 nt, from about 15 nt to about 30 nt or from about 15 nt to about 25 nt.

The dsRNA duplex of the Cas protein-binding segment of the guide nucleic acid can have a length from about 6 base pairs (bp) to about 50 bp. For example, the dsRNA duplex of the protein-binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp.

In some embodiments, the dsRNA duplex of the Cas protein-binding segment can have a length of 36 base pairs. The percent complementarity between the nucleotide sequences that hybridize to form the dsRNA duplex of the protein-binding segment can be at least about 60%. For example, the percent complementarity between the nucleotide sequences that hybridize to form the dsRNA duplex of the protein-binding segment can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the protein-binding segment is 100%.

The linker (e.g., the sequence that links a crRNA and a tracrRNA in a single guide nucleic acid) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a DNA-targeting RNA is 4 nt.

In some embodiments, the at least one guide RNA polynucleotide can bind to at least a portion of the mammalian genomes, mammalian genes, human genomes, or human genes. In some cases, the at least one guide RNA polynucleotide is capable of forming a complex with the nuclease to direct the nuclease to target the portion of the mammalian genomes, mammalian genes, human genomes, or human genes. In some embodiments, the at least one guide RNA polynucleotide can be complementary and bind to the mammalian genomes, mammalian genes, human genomes, or human genes as described herein.

In some embodiments, a nucleic acid sequence encoding one or more guide nucleic acids comprises a sequence having at least 90% identity (e.g., at least 91%, at least 92%, at least 93%, at least 94%, at least 95%, at least 96%, at least 97%, at least 98%, at least 99%, or 100% identity) to any one of SEQ ID NOs: 1-3. In some embodiments, a guide RNA useful in the compositions an methods provided herein comprises a sequence having at least 90% identity to any one of SEQ ID NOs: 1-3.

| SEQ ID | | Genomic Location | |
|---|---|---|---|
| NO | ID | GRCh37/hg19 | sgRNA sequence |
| 1 | RNA3 | chr4 90759274-90759294 | ACTTCTGATTCTCGTTGCCCT |
| 2 | RNA4 | chr4 90758135-90758155 | CACCCTCGTGAGCGGAGAACT |
| 3 | RNA6 | chr4 90758108-90758129 | GTGGCCATTCGACGACAGGTTA |

In some embodiments, the fusion proteins provided herein are targeted to alpha-synuclein. In some embodiments, the fusion proteins are targeted within the alpha-synuclein gene body. In some embodiments, the fusion proteins are targeted to a transcription regulatory region of the alpha-synuclein gene. Transcription regulatory regions include, but are not limited to, promoters and enhancers. In some embodiments, the fusion proteins are targeted via interaction with a guide RNA that is complementary or substantially complementary to a nucleic acid sequence in the alpha-synuclein gene or a transcription regulatory region thereof (e.g., a promoter, an enhancer, etc.). In some embodiments, the alpha-synuclein gene is a mammalian alpha-synuclein gene. In some embodiments, the alpha-synuclein gene is a human alpha-synuclein gene. In some embodiments, the alpha-synuclein gene is a non-human primate (e.g., rhesus, squirrel monkey, cynomolgus) alpha-synuclein gene.

In some embodiments, for example, as demonstrated in the Examples herein, binding of the fusion protein to the alpha-synuclein gene or a transcription regulatory region thereof results in decreased expression of the alpha-synuclein gene. The term “decreased expression.” as used herein, can refer to any reduction in a level of a gene product (e.g., messenger RNA or protein). In some embodiments, binding of the fusion protein to the alpha-synuclein gene or a transcription regulatory region thereof results in a gene expression decrease of up to 100% (e.g., up to 95%, up to 90%, up to 85%, up to 80%, up to 75%, up to 70%, up to 65%, up to 60%, up to 55%, up to 50%, up to 45%, up to 40%, up to 35%, up to 30%, up to 25%, up to 20%, up to 15%, or up to 10%) relative to gene expression of unbound alpha-synuclein genes.

### IV. DNA Constructs, Vectors, and Purified Viruses

Also provided herein is a DNA construct comprising a promoter operably linked to a recombinant DNA molecule described above. In addition to the definition provided in section (I) above, a nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter (for example, an antibiotic-inducible promoter such as a tetracycline-inducible promoter). In some embodiments, the promoter is a constitutive promoter (for example, the elongation factor 1 alpha, EF1-α, promoter). In some embodiments, the promoter is a human U6 promoter. In some embodiments, the promoter is a human MECP2 promoter. In some embodiments, the promoter is a human synapsin promoter. In some embodiments, the promoter is a human PGK promoter. In some embodiments, a provided DNA construct can comprise more than one promoter. In such embodiments, each promoter can be operably linked to a particular sequence of the DNA construct. For example, a DNA construct provided herein can comprise two promoters: a first promoter operably linked to the nucleic acid sequence that encodes a fusion protein comprising a CRISPR-associated nuclease and a transcriptional repressor; and a second promoter operably linked to the nucleic acid sequence that encodes a guide RNA that is complementary to a sequence in an alpha-synuclein gene or a gene regulatory region thereof. In some embodiments, the first promoter is a human MECP2 promoter, a human PGK promoter, or a human synapsin promoter. In some embodiments, the second promoter is a human U6 promoter.

The recombinant nucleic acids provided herein can be included in expression cassettes for expression in a host cell or an organism of interest. “Expression cassette” refers to a polynucleotide comprising a promoter or other regulatory sequence operably linked to a sequence encoding a protein, which can be, for example, specific for expression in the type of host cell (by making the promoter of the expression cassette host-cell specific, for example). The cassette will include 5′ and 3′ regulatory sequences operably linked to a recombinant nucleic acid provided herein that allows for expression of the modified polypeptide. The cassette may additionally contain at least one additional gene or genetic element to be cotransformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette will include in the 5′ to 3′ direction of transcription, a transcriptional and translational initiation region (i.e., a promoter), a polynucleotide disclosed herein, and a transcriptional and translational termination region (i.e., termination region) functional in the cell or organism of interest. The promoters described herein are capable of directing or driving expression of a coding sequence in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press. Cold Spring Harbor, N.Y.): Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, puromycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions. e.g., transitions and transversions, may be involved.

Further provided is a vector comprising one or more nucleic acids or expression cassettes set forth herein. The vector is contemplated to have the necessary functional elements that direct and regulate transcription of the inserted nucleic acid. These functional elements include, but are not limited to, one or more of a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid gene (See generally, Sambrook et al. Molecular Cloning: A Laboratory Manual, 4th ed., Cold Spring Harbor Laboratory Press, Cold Spring Harbor, 2012). The vector, for example, can be a plasmid.

There are numerous E. coli expression vectors known to one of ordinary skill in the art, which are useful for the expression of a nucleic acid. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide according to the present disclosure, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, genticin or G418 resistance, or other genes or phenotypes suitable for use as selectable markers, or methotrexate resistance for gene amplification. A number of suitable host cell lines capable of secreting intact human proteins have been developed in the art, and include CHO cells, HeLa cells, COS-7 cells, myeloma cell lines, Jurkat cells, etc. Expression vectors for these cells can include expression control sequences, such as an origin of replication, a promoter, an enhancer, and necessary information processing sites, such as ribosome binding sites. RNA splice sites, polyadenylation sites, and transcriptional terminator sequences.

The expression vectors described herein can also include the nucleic acids as described herein under the control of an inducible promoter such as the tetracycline inducible promoter or a glucocorticoid inducible promoter. The nucleic acids according to the present disclosure can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

In some embodiments, the vectors comprising the recombinant DNA molecules provided herein are viral vectors. Viral vectors have become the most widely used method for inserting genes into mammalian, e.g., human cells. Viral vectors, in some embodiments, are derived from lentivirus, pseudoviruses, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include an AAV1, AAV2, AAV4, AAV5, AAV6, AAV7. AAV8, or AAV9 serotype. In some instances, a viral vector is a chimeric viral vector, comprising viral portions from two or more viruses. In additional instances, the viral vector is a recombinant viral vector. In some embodiments, the viral vector is an adeno-associated virus vector. In some embodiments, the adeno-associated virus is an AAV9 serotype. In some embodiments, the viral vectors provided herein are up to about 5500 bp in length (e.g., up to about 5400 bp, up to about 5300 bp, up to about 5200 bp, up to about 5100 bp, up to about 5000 bp, up to about 4900 bp, up to about 4800 bp, up to about 4700 bp, up to about 4600 bp, or up to about 4500 bp). In some embodiments, as demonstrated in the Examples herein, the provided viral vectors of up to about 5100 bp in length (e.g., 5036 bp. 4796 bp) are able to be effectively delivered using AAV9, despite the generally known AAV kb packaging size limit (which is around 4.6 kb).

In some embodiments, a viral vector provided herein comprises a nucleic acid sequence having at least 70% identity (e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO:20 or SEQ ID NO: 21.

Also provided herein are isolated viruses comprising any of the viral vectors described herein. Methods for generating isolated viruses are known to one of skill in the art. In some embodiments, the isolated virus is an adeno-associated virus. In some embodiments, the isolated virus is an AAV9 serotype.

A host cell comprising a nucleic acid, a DNA construct, or a vector described herein is also provided. The host cell can be an in vitro, ex vivo, or in vivo host cell. Populations of any of the host cells described herein are also provided. A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook, Ausubel, and Berger (all supra), as well as Freshney (1994) Culture of Animal Cells, a Manual of Basic Technique, 3^{rd} Ed., Wiley-Liss, New York and the references cited therein: Doyle and Griffiths (1997) Mammalian Cell Culture: Essential Techniques John Wiley and Sons. NY; Humason (1979) Animal Tissue Techniques, 4^{th} Ed. W.H. Freeman and Company; and Ricciardelli, et al., (1989) In vitro Cell Dev. Biol. 25:1016-1024.

As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid, for example, a nucleic acid encoding a recombinant protein described herein, into a host cell.

Any of the proteins described herein can be purified or isolated from a host cell or population of host cells. For example, a recombinant nucleic acid encoding any of the proteins described herein can be introduced into a host cell under conditions that allow expression of the protein. In some embodiments, the recombinant nucleic acid is codon-optimized for expression. After expression in the host cell, the recombinant protein can be isolated or purified using purification methods known in the art. Furthermore, recombinant nucleic acids as described herein can contain tags useful for isolation, for example, His tags.

An “isolated” or “purified” polypeptide or protein is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, 1%, 0.5%, or 0.1% (total protein) of contaminating protein. When the protein of the present disclosure or its biologically active portion is recombinantly produced, optimally culture medium represents less than about 30%, 20%, 10%. 5%, 1%, 0.5%, or 0.1% (by concentration) of chemical precursors or non-protein-of-interest chemicals. In this manner, compositions may consist essentially of the isolated or purified polypeptide of interest.

### V. Pharmaceutical Compositions and Formulations

The recombinant DNA molecules provided herein, and the products encoded by said recombinant DNA molecules, are suitable for a variety of administration routes in vitro or in vivo. Compositions comprising a recombinant DNA molecule of the present disclosure and a pharmaceutically acceptable carrier (excipient) are provided. In some embodiments, the compositions comprise a viral vector and/or purified virus, as described above. A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The pharmaceutical compositions may further comprise a diluent, solubilizer, emulsifier, preservative, and/or adjuvant to be used with the methods disclosed herein. Such pharmaceutical compositions can be used in a subject that would benefit from administration of any of the recombinant DNA molecules or fusion proteins described herein, for example, a subject having or suspected to have Parkinson's disease, or any subject in which a reduction of alpha-synuclein gene expression is desirable.

Suitable carriers and their formulations are described in Remington: The Science and Practice of Pharmacy, 21^{st} Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous and/or intravenous administration. In certain embodiments, the formulation comprises an appropriate amount of a pharmaceutically-acceptable salt to render the formulation isotonic. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCL, citrates, phosphates or other organic acids): bulking agents (such as mannitol or glycine); chelating agents (such as ethylenediamine tetraacetic acid (EDTA)): complexing agents (such as caffeine, polyvinylpyrrolidone, beta-cyclodextrin or hydroxypropyl-beta-cyclodextrin); fillers: monosaccharides, disaccharides, and other carbohydrates (such as glucose, mannose or dextrins): proteins (such as serum albumin, gelatin or immunoglobulins); coloring, flavoring and diluting agents: emulsifying agents: hydrophilic polymers (such as polyvinylpyrrolidone); low molecular weight polypeptides; salt-forming counterions (such as sodium): preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol): sugar alcohols (such as mannitol or sorbitol): suspending agents; surfactants or wetting agents (such as pluronics. PEG, sorbitan esters, polysorbates such as polysorbate 20, polysorbate 80, triton, tromethamine, lecithin, cholesterol, tyloxapal): stability enhancing agents (such as sucrose or sorbitol); tonicity enhancing agents (such as alkali metal halides, preferably sodium or potassium chloride, mannitol sorbitol); delivery vehicles: diluents: excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22^{nd} Edition, Lloyd V. Allen, Jr., ed., The Pharmaceutical Press (2014). In certain embodiments, such compositions may influence the physical state, stability, rate of in vivo release and/or rate of in vivo clearance of the recombinant DNA molecules described herein.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising a recombinant DNA molecule or fusion protein disclosed herein can be prepared for storage by mixing the selected composition having the desired degree of purity with optional formulation agents (see Remington: The Science and Practice of Pharmacy, 22^{nd} Edition, Lloyd V. Allen, Jr., ed., The Pharmaceutical Press (2014)) in the form of a lyophilized cake or an aqueous solution. Further, in certain embodiments, a composition comprising a recombinant DNA molecule or fusion protein disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions or suspensions, including saline and buffered media) comprising a recombinant DNA molecule or fusion protein in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product that can then be delivered via a depot injection. In certain embodiments, hyaluronic acid can also be used, and can have the effect of promoting sustained duration in the circulation.

In certain embodiments, the compositions can be selected for inhalation (such as nasal inhalation, for example) or for delivery through the digestive tract, such as orally. Compositions for oral administration include powders or granules, suspension or solutions in water or non-aqueous media, capsules, sachets, or tables. Thickeners, flavorings, diluents, emulsifiers, dispersing aids or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5.

In certain embodiments, a pharmaceutical composition can comprise a therapeutically effective amount of a recombinant DNA molecule or fusion protein in a mixture with non-toxic excipients suitable for the manufacture of tablets, aqueous formulations, or aerosols for inhalation. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia: or lubricating agents such as magnesium stearate, stearic acid, or talc.

Additional pharmaceutical compositions can be selected by one skilled in the art, including formulations involving a recombinant DNA molecule or fusion protein in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. Sce for example. International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (sec. e.g., U.S. Pat. Nos. 3,773,919; 5,594,091; 8,383,153; 4,767,628; International Application Publication No. WO1998043615, Calo, E. et al. (2015) Eur. Polymer J 65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15:167-277; and Langer (1982) Chem Tech 12:98-105), ethylene vinyl acetate (Hsu and Langer (1985) J Biomed Materials Res 19 (4): 445-460) or poly-D(−)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692: European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In still another aspect, unit dose forms comprising a recombinant DNA molecule as described in this disclosure are provided. A unit dose form can be formulated for administration according to any of the routes described in this disclosure. In one example, the unit dose form is formulated for intravenous or intraperitoneal administration. In still another aspect, pharmaceutical packages comprising unit dose forms of a recombinant DNA molecule described herein are provided.

The recombinant DNA molecules and fusion proteins disclosed herein are ideally suited for the preparation of a kit. In some embodiments, kits are provided for carrying out any of the methods described herein. The kits of this disclosure may comprise a carrier container being compartmentalized to receive in close confinement one or more containers such as vials, tubes, syringes, and the like, each of the containers comprising one of the separate elements to be used in the method.

A recombinant DNA molecule or fusion protein as described in this disclosure for use in treating a subject may be delivered in a pharmaceutical package or kit to doctors and subjects. Such packaging is intended to improve patient convenience and compliance with the treatment plan. Typically the packaging comprises paper (cardboard) or plastic. In some embodiments, the kit or pharmaceutical package further comprises instructions for use (e.g., for administering according to a method as described herein).

In one embodiment, the kit or pharmaceutical package comprises a DNA molecule or fusion protein in a defined, therapeutically effective dose in a single unit dosage form or as separate unit doses. The dose and form of the unit dose (e.g., tablet, capsule, immediate release, delayed release, etc.) can be any doses or forms as described herein.

In one embodiment, the kit or pharmaceutical package includes doses suitable for multiple days of administration, such as one week, one month, three months, or six months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### VI. Methods of Treatment

Also provided herein are methods of treating a subject (otherwise referred to herein as a “subject in need” or a “subject in need thereof”), comprising administering to the subject a therapeutically effective amount of a pharmaceutical composition as described above. In some embodiments, the subject has Parkinson's disease (e.g., as diagnosed by methods known in the art). In some embodiment, the subject is suspected to have Parkinsons's disease. In some embodiments, the subject has a non-Parkinson's disease disorder that is at least partially caused by excess expression or accumulation of alpha-synuclein.

The term “normal” as used in the context of “normal cell,” is meant to refer to a cell of an untransformed phenotype or exhibiting a morphology of a non-transformed cell of the tissue type being examined.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators.” and “modulators” of expression or of activity are used to refer to inhibitory, activating, or modulating molecules, respectively, identified using in vitro and in vivo assays for expression or activity of a described target protein or cell type (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Activators can also increase activity and/or proliferation of immune cells and glial cells (such as microglial in the central nervous system, for example). Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25%, 10%, 5% or 1%. Activation of the described target protein is achieved when the activity value relative to the control is 110%, optionally 150%, optionally 200%, 300%, 400% 500%, or 1000-3000% or more higher.

The term “administered continuously” refers to the continuous delivery of a therapeutic agent, e.g., compound, molecule, peptide, biologic, chemical, etc. over a 24-hour period.

As used herein, the terms “pharmaceutically acceptable” or “pharmacologically acceptable” refer to compositions that do not substantially produce adverse reactions, e.g., toxic, allergic, or immunological reactions, when administered to a subject.

As used herein, the term “subject” means a mammalian subject. Exemplary subjects include, but are not limited to humans, monkeys, dogs, cats, mice, rats, cows, pigs, birds, horses, camels, goats, and sheep. The terms “individual.” “subject.” “host.” and “patient,” used interchangeably herein and refer to any mammalian subject for whom diagnosis, treatment, or therapy is desired, particularly humans.

As used herein, “treating” or “treatment” of any disease or disorder refers to preventing or ameliorating a disease or disorder in a subject or a symptom thereof. The term ameliorating refers to any therapeutically beneficial result in the treatment of a disease state, e.g., Parkinson's disease, lessening in the severity or progression, or curing thereof. Thus, treating or treatment cam include ameliorating at least one physical parameter or symptom. Treating or treatment can include modulating the disease or disorder, either physically (e.g., stabilization of a discernible symptom) or physiologically (e.g., stabilization of a physical parameter) or both. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating Parkinson's disease in a subject by administering a composition as described in this disclosure is considered to be a treatment if there is a 10% reduction in one or more symptoms of Parkinson's disease in the subject as compared to a control. Thus the reduction can be a 10%, 20%, 30%, 40%, 50%, 60%, 70%, 80%, 90%, 100%, or any percent reduction in between 10% and 100% as compared to native or control levels. In some embodiments, the compositions described herein are administered to the subject until the subject exhibits amelioration of at least one symptom of Parkinson's disease. It is understood that treatment does not necessarily refer to a cure or complete ablation of the disease, condition, or symptoms of the disease or condition.

“Administering” or “administration of” a composition to a subject (and grammatical equivalents of this phrase), as used herein, refer to the act of injecting or otherwise physically delivering a substance as it exists outside the body (e.g., a recombinant DNA molecule or fusion protein composition provided herein) into a subject. Administration can be via enteral or parenteral routes. In some embodiments, administration is by mucosal, intradermal, intravenous, intramuscular, subcutaneous delivery and/or any other method of physical delivery described herein or known in the art. In some embodiments, the composition is administered intrathecally. In some embodiments, the composition is administered into the cisterna magna. In some embodiments, the composition is administered into the cerebrospinal fluid. In some embodiments, as demonstrated in the Examples herein, intrathecal delivery of a viral vector as described herein results in penetration of virus and gene downregulation in deep brain regions.

Administration refers to direct administration, which may be administration to a subject by a medical professional or may be self-administration, and/or indirect administration, which may be the act of prescribing a composition. For example, a physician who instructs a subject to self-administer a composition and/or provides a subject with a prescription for a composition is administering the composition to the subject.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing polypeptides are well known to those of skill in the art. Generally, such systems should utilize components that will not significantly impair the biological properties of the recombinant DNA molecules or fusion proteins (see, for example, Remington's Pharmaceutical Sciences, 18th edition, 1990, Mack Publishing). Those of skill in the art can readily determine the various parameters and conditions for producing injectates or infusates without resorting to undue experimentation. Administration can be achieved by, e.g., topical administration, local administration, injection, by means of an implant.

As used herein, the term “therapeutically effective amount” refers to an amount of recombinant DNA molecule or fusion protein composition as described herein that, when administered to a subject, is effective to achieve an intended purpose, e.g., to treat Parkinson's disease. A therapeutically effective amount is not, however, a dosage so large as to cause adverse side effects. A therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics or treatments that are administered to the subject. Although individual needs may vary, determination of optimal ranges for effective amounts of formulations is within the skill of the art. Human doses can be extrapolated from animal studies. Generally, the dosage required to provide an effective amount of a formulation, which can be adjusted by one skilled in the art, will vary depending on the age, health, physical condition, weight, type and extent of the disease or disorder of the recipient, frequency of treatment, the nature of concurrent therapy (if any), the method of administration, and the nature and scope of the desired effect(s) (Nies et ah, Chapter 3 In: Goodman & Gilman's The Pharmacological Basis of Therapeutics, 9th Ed., Hardman et ah, eds., McGraw-Hill, New York, NY, 1996). It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication.

In some embodiments, the recombinant DNA molecule or fusion protein composition is administered to the subject at least once a day, at least twice a day, or at least three times a day. In some embodiments, the composition is administered on consecutive days or on non-consecutive days. In some instances, the composition is administered to the subject for at least 1 day, at least 2 days, at least 4 days, at least 5 days, at least 6 days, at least 1 week, at least 2 weeks, at least 3 weeks, at least 1 month, at least 2 months, at least 3 months, at least 4 months, at least 5 months, or at least 6 months.

A pharmaceutical preparation as described herein can comprise a therapeutically effective amount of a recombinant DNA molecule or fusion protein composition described herein. Such effective amounts can be readily determined by one of ordinary skill in the art as described above. Considerations include the effect of the administered DNA molecule, or the combinatorial effect of the recombinant DNA molecule with one or more additional active agents, if more than one agent is used in or with the pharmaceutical composition.

Suitable human doses of any of the compositions described herein can further be evaluated in, e.g., Phase I dose escalation studies. See, e.g., van Gurp et al. (2008) Am J Transplantation 8 (8): 1711-1718; Hanouska et al. (2007) Clin Cancer Res 13 (2, part 1): 523-531; and Hetherington et al. (2006) Antimicrobial Agents and Chemotherapy 50 (10): 3499-3500.

Toxicity and therapeutic efficacy of the recombinant DNA molecule and fusion protein compositions described herein can be determined by known pharmaceutical procedures in cell cultures or experimental animals (e.g., animal models of any of the disease states described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A recombinant DNA molecule or fusion protein composition that exhibits a high therapeutic index is preferred. While constructs that exhibit toxic side effects may be used, care should be taken to design a delivery system that targets such constructs to the site of affected tissue and to minimize potential damage to normal cells and, thereby, reduce side effects.

The data obtained from the cell culture assays and animal studies can be used in formulating a range of dosage for use in humans. The dosage of a recombinant DNA molecule or fusion protein composition lies generally within a range of circulating concentrations of the recombinant DNA molecule or fusion protein compositions that includes the ED₅₀ with little or no toxicity. The dosage may vary within this range depending upon the dosage form employed and the route of administration utilized. For compositions described herein, the therapeutically effective dose can be estimated initially from cell culture assays. A dose can be formulated in animal models to achieve a circulating plasma concentration range that includes the EC₅₀ (i.e., the concentration of the construct—e.g., polypeptide—that achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

In some embodiments, a recombinant DNA molecule or fusion protein composition described herein can be administered to a subject as a monotherapy. Alternatively, the recombinant DNA molecule or fusion protein composition can be administered in conjunction with other therapies for Parkinson's disease (for example small molecule therapies such as levodopa and derivatives thereof). For example, the composition can be administered to a subject at the same time, prior to, or after, a second therapy. In some embodiments, the recombinant DNA molecule or fusion protein composition and the one or more additional active agents are administered at the same time. Optionally, the recombinant DNA molecule or fusion protein composition can be administered first in time and the one or more additional active agents are administered second in time. In some embodiments, the one or more additional active agents are administered first in time and the recombinant DNA molecule or fusion protein composition is administered second in time. Optionally, the recombinant DNA molecule or fusion protein composition and the one or more additional agents can be administered simultaneously in the same or different routes. For example, a composition comprising the recombinant DNA molecule optionally contains one or more additional agents.

Monitoring a subject (e.g., a human patient) for an improvement of a Parkinson's disease symptom, as defined herein, means evaluating the subject for a change in a disease parameter. e.g., a reduction in one or more symptoms of Parkinson's disease exhibited by the subject. In some embodiments, the evaluation is performed at least one (1) hour, e.g., at least 2, 4. 6, 8. 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment: or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality. e.g., adding or dropping any of the treatments for a viral infection described herein.

Disclosed herein are materials, compositions, and methods that can be used for, can be used in conjunction with, or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are various additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### Examples

The following examples are offered to illustrate, but not to limit, aspects of the present disclosure.

### Example 1. Design and Characterization of sgRNAs for Downregulation of Alpha-Synuclein

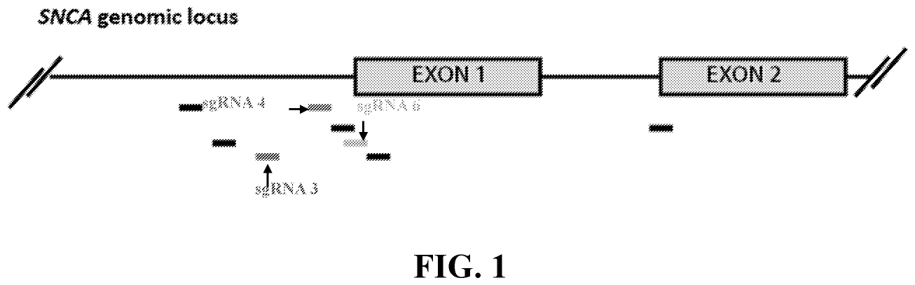

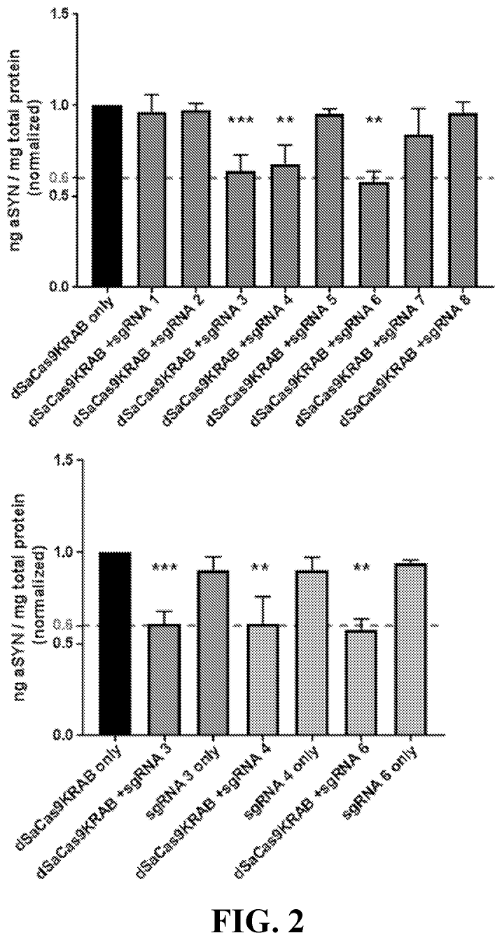

A combination of manual sgRNA design and use of the prediction program CRISPOR (Concordet and Haeussler, 2018, Nucleic Acids Research 46: W242-W5) was used to design sgRNAs for CRISPR/Cas9-mediated downregulation. It is known that the alpha-synuclein promoter's transcriptional start site 2 (TSS2) (Heman-Ackah et al., 2016. Sci. Rep. 6:28420) supports alpha-synuclein downregulation using S. pyogenes Cas9. Several small guide RNAs (sgRNA) were designed around TSS2 with a PAM sequence NNGRRT that also show high homology to non-human primates (rhesus and cynomolgus) (FIG. 1). Three of these designed sgRNAs (sgRNA 3, sgRNA 4, and sgRNA 6) showed more than 40% reduction of alpha-synuclein protein after transient transfection in HEK293 cells 72 hrs after transfection (FIG. 2). The sequence of sgRNA 3 is ACTTCTGATTCTCGTTGCCCT (SEQ ID NO:1). The sequence of sgRNA 4 is CACCCTCGTGAGCGGAGAACT (SEQ ID NO:2) The sequence of sgRNA 6 is GTGGCCATTCGACGACAGGTTA (SEQ ID NO:3). It is noted in this embodiment that the human U6 promoter prefers a ‘G’ at the transcription start site to have high expression, so adding this G (at the 5′ of SEQ ID NO:3) can help with expression. In other embodiments, this “G” at the 5′ end is optional and it is possible for the plasmid to still express without the G. Alternatively, if the spacer sequence between the human U6 promotor and the sgRNA starts with a ‘G’, there is naturally this “G” present on the 5′ end of the sgRNA (as the 3′ end of the spacer) and one does not need to add an additional ‘G’ to the sgRNA sequence.

sgRNA 6 was used for in vivo experimentation because of its 100% homology to non-human primate alpha-synuclein promoter region (rhesus, squirrel monkey, and cynomolgus), because it has no predicted off-targets in the human genome, and because it showed a 50% downregulation in HEK cell experiments.

### Example 2. Design and Characterization of Adeno-Associated Expression Cassette

Since neurodegeneration in Parkinson's disease affects neurons primarily in the central and peripheral nervous system, tissue-specific expression of the Cas9 transgene in neurons was employed to reduce toxicity and unwanted expression in other tissues and organs and improve the overall health safety profile of a therapeutic vector. Two neuron-specific short promoters, the SYNAPSIN (469 bp) (McLean et al . . . 2014, Neuroscience Letters 576:73-78) and MECP2 (229 bp) (Adachi ct al., 2005, Hum. Mol. Genet. 14:3709-22) promoters, were tested. Compared to ubiquitous promoters such as CMV or CAG promoters, these short promoters allow fitting within the packaging capacity of 4.8 kb of an AAV vector.

When downregulation does not have the desired effect in neurons, it is also feasible to use a ubiquitous promoter like phosphoglycerate kinase (511 bp) to achieved more widespread expression in neurons and non-neuronal cells of the brain.

S. aureus Cas9 (saCas9) has a length of 3.2 kb and is about 1 kb shorter than the more commonly used S. pyogenes Cas9. For the AAV cassette design, the smaller Cas9 with two mutations (D10A and N580A) that abolish its nuclease activity (Ran et al., 2015, Nature 520:186-91) was used. The saCas9 sequence is flanked with nuclear localization signals to shuttle Cas9 to the nucleus. To repress gene expression, saCas9 is flanked by two Krueppel-associated (KRAB) domains.

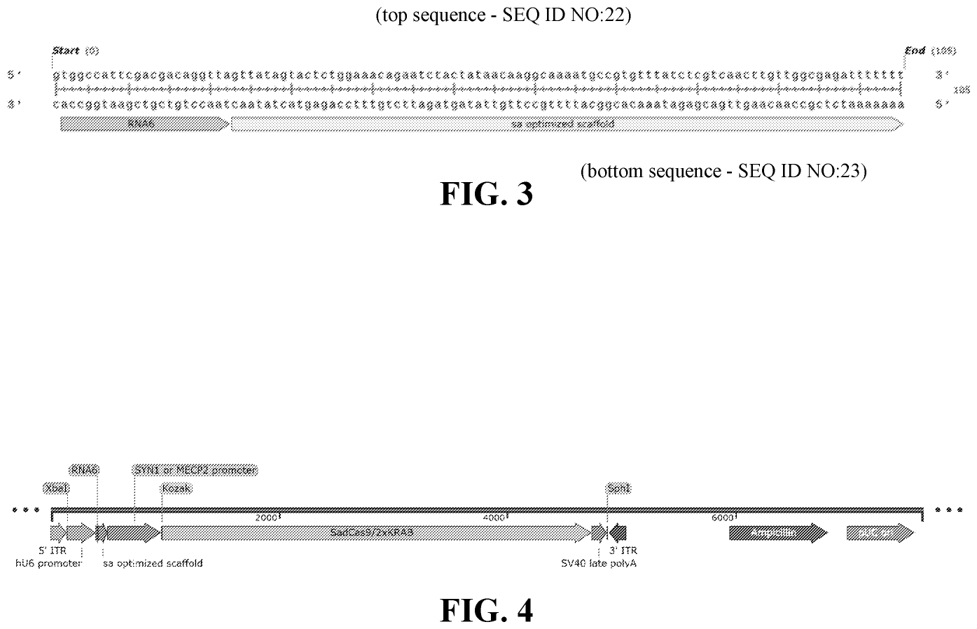

The human U6 promoter>small guide RNA6 construct was placed upstream of the saCas9 constructs. The human U6 promoter is 249 bp in length and is shorter than the mouse U6 promoter by 65 bp. A guanosine nucleotide follows the human U6 promoter to initiate transcription, then the sequence for sgRNA6 and S. aureus optimized scaffold for binding to the alpha-synuclein promoter target (FIG. 3).

A challenge with AAV genomes is their small packaging capacity of 4.6 kb, including the ITR repeats. Despite use of small promoter elements and S. aureus Cas9, the size of the expression cassette is over the limit for the MECP2 promoter construct of 4796 bp and for the SYN1 promoter construct 5036 bp (FIG. 4). However, sufficient virus titers and successful delivery of the virus were achieved in vivo, as described below.

### Example 3. Adeno-Associated Virus Mediated In Vivo Delivery

AAV9 was used as an in vivo delivery vector. AAV9, which has been well-investigated for delivering transgenes, is a compelling candidate for gene therapy in humans. In previous clinical trials, AAV9 showed a favorable safety profile. AAVs are small viruses (25 nm) of the Parvoviridae family. These viruses have a non-enveloped capsid that stores genetic material in the form of single-stranded DNA. AAVs can infect dividing and nondividing cells. These characteristics make them potential agents for administering gene therapy to post-mitotic cells such as neurons. Based on the capsid protein composition, 13 AAV (AAV1-AAV13) serotypes show differences in their tropism and transduction efficiencies across various tissues. These differences across multiple AAV serotypes allow targeting particular specialized cells selectively. Of all the identified serotypes. AAV9 has the highest tropism for the central nervous system (Pattali et al., 2019, Gene Therapy 26:287-95).

For recombinant AAV manufacturing, the transfer plasmid carrying the Cas9 expression cassette described above is co-transfected with the Rep-cap plasmid and helper plasmid encoding adenovirus genes that mediate AAV replication into HEK293T packaging cells. After the incubation period, viral particles are harvested from the cell lysate or supernatant and concentrated by poly(ethylene glycol) precipitation. For ultra-purified AAV, viral particles are concentrated by cesium chloride gradient ultracentrifugation. High titers of at least 1013 gc/ml were produced. Titer was determined by quantitative PCR method. Viral DNA was extracted from viral particles. The ITR sequence of AAV2 was used as a target to quantify the amount of viral genome. The virus was sterility and mycoplasma tested.

For widespread delivery in the central nervous system and minimal systemic distribution, intrathecal delivery of the gene vector via cisterna magna was used (Lukashchuk et al . . . 2016, Mol. Ther. Methods Clin. Dev. 3:15055: Taghian et al., 2020. Mol. Ther. 28:411-21). This supports downregulation of the alpha-synuclein throughout the central nervous system in neurons, but should reduce complications from systemic delivery of AAV9 and its expression in non-CNS tissues such as liver and heart, which could lead to toxicity. Such a delivery method is expected to reduce success in clinical trials.

All experiments involving mice were conducted according to animal welfare regulations under protocol Stanford protocol APLAC-33261. Animals were maintained in a controlled facility in a 12-hour dark/12-hour light cycle, a standardized room temperature (RT) of 21° C., with free access to food and water.

Mice were anesthetized in an induction chamber with 3-4% isoflurane, and a nose cone to sustain isoflurane anesthesia at 1-2% was used throughout the surgery. When fully anesthetized, the animals were placed in a stereotaxie frame on a temperature-controlled heating pad and ophthalmic ointment was applied to keep eyes moist during surgery and prevent cornea damage. T head and neck of the mouse were shaved with #40 size clipper blades, then small hair particles were removed with cellophane tape and exposed skin was cleaned with a 70% alcohol and then two times with iodine solution (2%). For post-surgical analgesia, buprenorphine SR (0.5 mg/kg) was administered subcutaneously. Position of the head of the mouse in the stereotaxic frame was assured by lifting the posterior edge of the stereotactic frame to form a 30° angle with the table surface. The tooth bar was used to press the head down on the naison. The skin over the occipital crest was lifted with tweezers, and an almond-shaped piece of skin of approximately 1 cm was cut along the midline. Using the occipital crest as a reference point, the superficial connective tissue was pulled apart to expose the neck muscles below. The muscles were separated at the midline by carefully running the forceps down the middle of the incision site in the anterior-to-posterior axis. Using a surgical eye spear or cotton swab, the dural membrane covering the cisterna magna was wiped. Using a preloaded 10 ul Hamilton syringe and a 30-gauge needle, the virus (10 microliter at a concentration of 1.52×1010 gc/microliter at a rate of 1 μl/minute) was injected into the cisterna magna by inserting the syringe at an angle of 45° relative to the mouse head 1 mm into the cisterna magna. The wound was closed with non-absorbable 5-0 suture. Mice recovered in a temperature-controlled recovery cage. Four and twenty-four weeks after surgery, animals were terminally anesthetized and euthanized with isoflurane (induction 2%, euthanasia 6-8%), and cardiac perfusion was performed with 100 ml of cold (4° C.) PBS (0.1 M, pH 7.2), placing a 0.50 mm×16 mm syringe through the left ventricle of the heart to remove blood from the brain and organs. Tissues were harvested and flash-frozen in liquid nitrogen.

For in vivo experiments, a previously established humanized mouse model for alpha-synuclein Dbl-PAC-Tg (SNCAA53T): Snca−/− on a mixed genetic background 129S6/SvEvTac*FVB/N and mouse alpha-synuclein knockout (Kuo et al., 2010, Hum. Mol. Genet. 19:1633-50; informatics.jax.org/allele/MGI: 4412066) was used. The model expresses the human alpha-synuclein gene solely under the genomic regulatory elements of the human SNCA promoter (34 kb upstream of the SNCA gene and all intronic regions that allows splicing). The PAC-Tg (SNCA^{A53T}) transgene located on the 146 kb RPCI-1 human male P1 artificial chromosome (PAC) clone 27M07 and contains the entire human SNCA (synuclein, alpha (non A4 component of amyloid precursor)) gene and 34 kb of its upstream region and have the A53T human mutation. The transgene was microinjected into the pronuclei of fertilized FVB/N ova. The targeting vector for the Snca knockout allele was designed to replace exons 4 and 5 with a reverse-oriented neomycin resistance cassette. The construct was electroporated into 129S6/SvEvTac-derived TC-1 embryonic stem (ES) cells. Correctly targeted ES cells were injected into recipient blastocysts and the resulting chimeric mice were crossed to 129S6/SvEvTac. The transgenic dbl-PAC-Tg (SNCA^{A53T}) strains and the Snca−/− strain were combined to form a homozygous double transgenic, homozygous Scena KO strain maintained on a mixed FVB/N; 129S6/SvEvTac background for control experiments.

### Example 4. Characterization of In Vivo Alpha-Synuclein Downregulation

Before tissue sectioning, the frozen brain tissue blocks were equilibrated at −20° C., for at least 1 hour in a cryostat. The blocks were sectioned by cutting 15 μm thick coronal sections. The sections were mounted onto SuperFrost Plus slides (Fisher Scientific #12-550-15). After mounting, the sections were kept in the cryostat for 2 hours at −20° C. Slides with tissue sections were stored at −80° C., up to 3 months or −20° C., up to 1 month until further processed for imaging.

In situ hybridization RNAScope was performed according to manufacturer's instructions using the RNAscope multiplex fluorescent assay v.2 kits (ACD Systems, Cat. No. 323100). In brief, tissue sections from −80° C. were placed immediately in pre-chilled 10% neutral buffered formalin for 15 min at 4° C. After fixation, tissue sections were dehydrated gradually in 50% ethanol (5 min, RT), in 70% ethanol (5 min, RT), and then in twice in 100% ethanol (5 min. RT). Sections were air-dried for 5 min after ethanol treatment. Tissue sections were treated with RNAScope hydrogen peroxide (Part Number 322335) for 10 min at RT, followed by a protease IV incubation (30 min, RT) inside a Hybez humidity control tray wet with distilled water. Hybridization probes: Hs-SNCA (ACD Systems, Cat. No. 605681-C1), Mm-Rbfox3 (NeuN)-C2 (1:600) (ACD Systems, Cat. No. 313311-C2), saCas9-C3 (1:600) (ACD Systems, Cat. No. 501621-C3), positive control (ACD Systems, Cat. No. 320861) and negative control (ACD Systems, Cat. No. 320871) were performed in a hybridization oven (ACD, HybEZ™ II Hybridization System, ACD Systems, Cat. No. 321710) at 40° C., for 2 hours. Slides were washed and underwent three steps of amplification: AMP1 (30 min, 40° C.), AMP2 (40° C.), and AMP3 (15 min, 40° C.). Then, slides were incubated with RNAScope HRP-C1 for 15 min at 40° C.′ followed by a washing cycle and incubation with TSA Plus fluorophore (1:600) for 30 min at 40° C. Lastly, slides were blocked with an HRP blocker for 15 min at 40° C., and counterstained with a nuclear marker (DAPI). RNAScope slides were covered with Prolong Gold Antifade reagent (Invitrogen. P36930) and a 22×50 mm coverslip, then sealed with nailpolish before imaging.

Downregulation of a-syn was quantified for all slides using the CellReporter Xpress® software (Molecular devices version 2.6.130) to detect the fluorescent signal from the RNAScope probe for alpha synuclein mRNA as “small pits” (rounded small fluorescent signal) and “large pits” (accumulation of fluorescent signal). This analysis result was the number of small or large pits in each individual cell. Then, the pit count were binned into 5 different classifications (0-4 bin): bin 0, 0 pits or vesicles per cell, bin 1 to a count of 1-3 pits or vesicles per cell, bin 2 to 4-9 pits or vesicles per cell, bin 3 to counts of 10-15 pits or vesicles per cell, and bin 4 corresponding to all counts higher than 15 pits or vesicles per cell. Then, H-scores were calculated using this binning system via the formula:

$${\sum }_{n = 0}^{4} \frac{Vesicle or pit Count}{Total Cell Count} \times n \times 100 \qquad ( eq . 1 )$$

the sum of vesicle count divided by total cell count multiplied by the percentage value assigned to a particular bin. Transduction efficacy was calculated as the pit sum from Bins 1 to 4. After H-Score quantification and organization, the data were analyzed with GraphPad Prism®. Statistical analysis of RNA foci counts was performed in GraphPad Prism® using a one-way ANOVA (α≤0.05) and a Brown-Forsythe & Welch test (95% Cl).

### Example 5. Cisterna Magna Delivery of AAV9 Virus Vector Allows Efficient Transduction Throughout the Mouse Brain for Both MECP2 and SYN1 Promoter

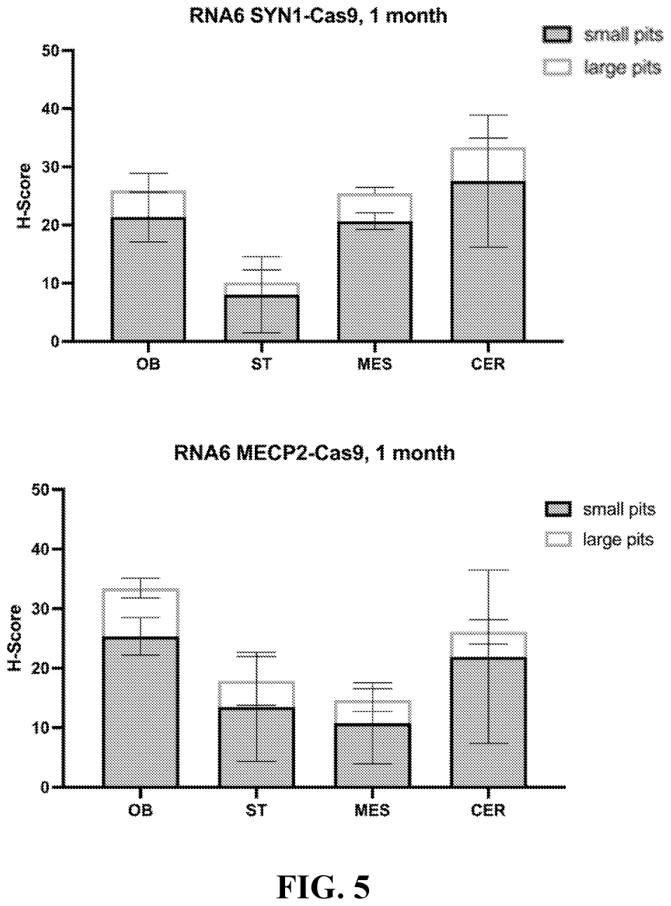

One month after AAV cisterna magna surgery, brains were collected and analyzed for Cas9 expression using in situ hybridization RNAScope in coronal sections of the olfactory bulb (OB), striatum (ST), mesencephalon (MES), and cerebellum (CER) (FIG. 5), Cas9 shows robust expression across all brain regions, illustrating that intrathecal injection via cisterna magna is a suitable route of delivery for the AAV9 viral vector to express the target gene, Cas9 expression is detected in cells that also co-expression neuronal nuclear protein (NeuN), a neuron-specific marker, which is expected as Cas9 expression is driven by neuron-specific promoters MECP2 and SYN1. Comparable expression was observed for both expression cassettes.

### Example 6. AAV9-Mediated Cas9 Expression Facilitates Significant Alpha-Synuclein Downregulation Via Small Guide RNA 6

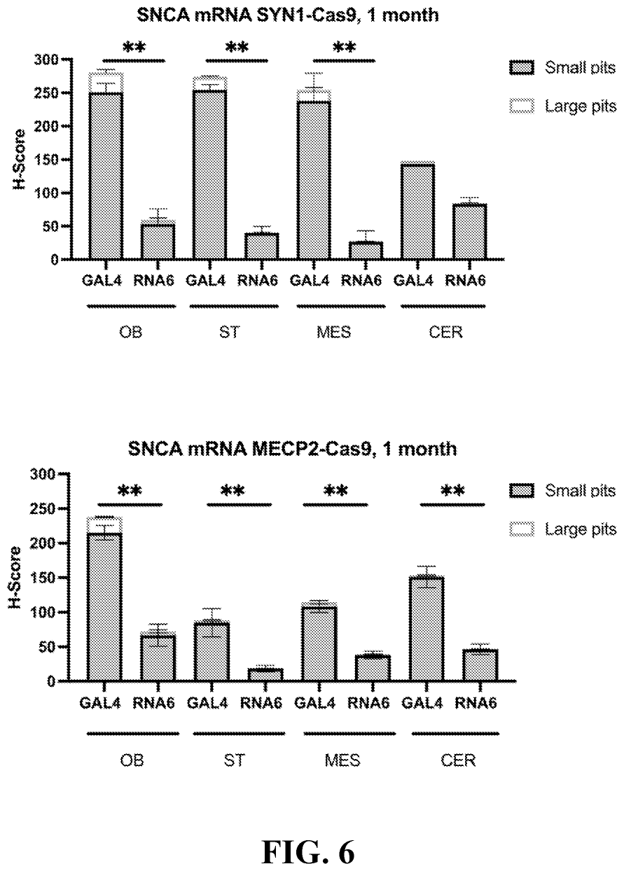

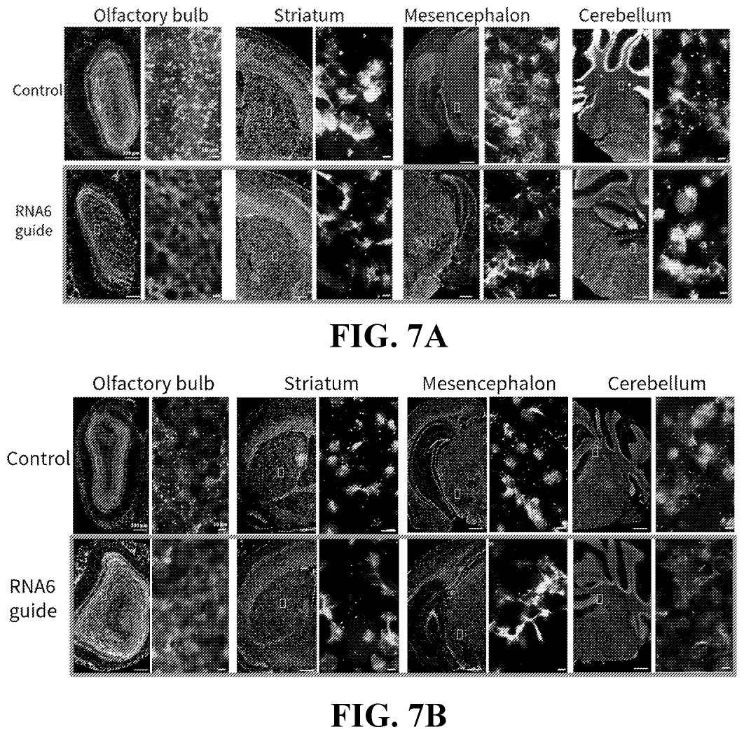

One month after AAV cisterna magna surgery, brains were collected and analyzed for alpha-synuclein expression using in situ hybridization RNAScope in coronal sections of the olfactory bulb (OB), striatum (ST), mesencephalon (MES), and cerebellum (CER) and compared between the TSS2 targeting small guide RNA and non-sense Gal4 small guide RNA (FIG. 6; FIGS. 7A-7B). Up to 80% downregulation of alpha-synuclein was detected for RNA6 compared to Gal4 control. Both MECP2 and SYN1 promoters showed a comparable effect in alpha-synuclein downregulation.

These data show proof of concept that AAV9 can deliver Cas9 successfully via intrathecal administration and that small guide RNA6 mediates robust alpha-synuclein downregulation in vivo.

### Example 7. AAV9-Mediated Cas9 Expression Modulates Microglial Activation and SNCA Expression Via Small Guide RNA 2

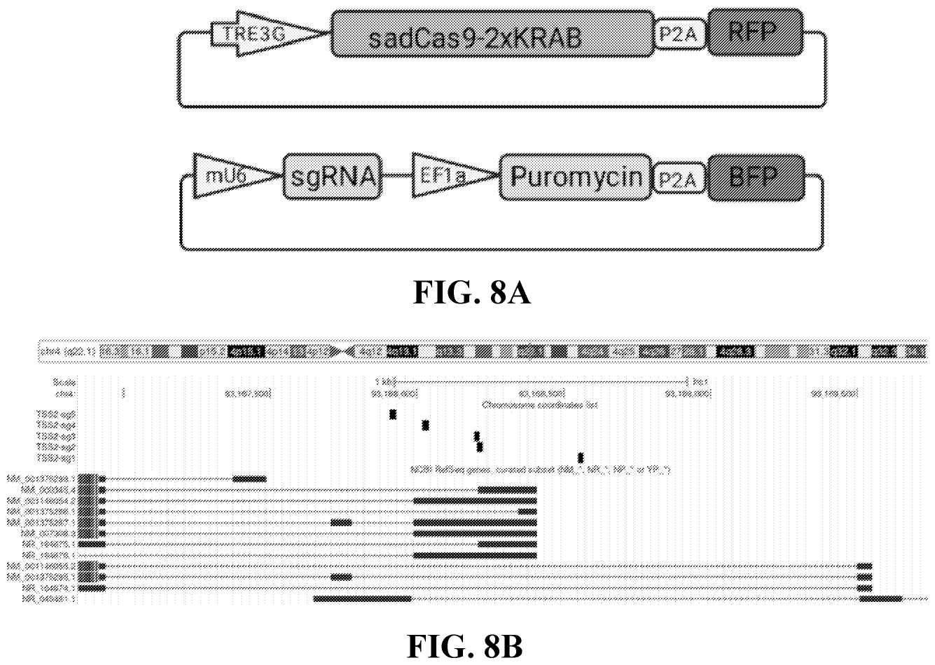

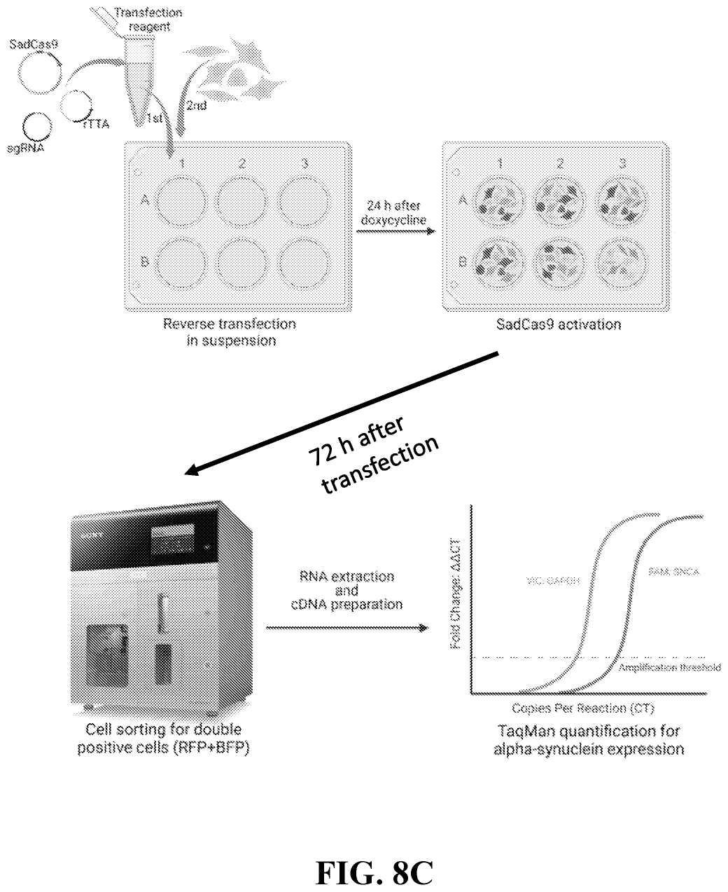

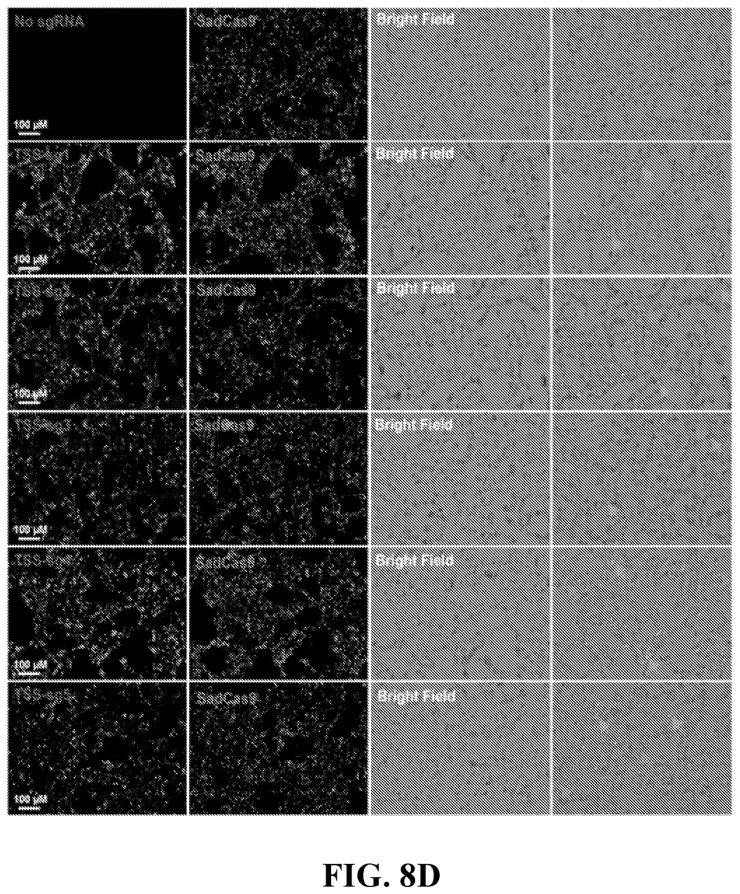

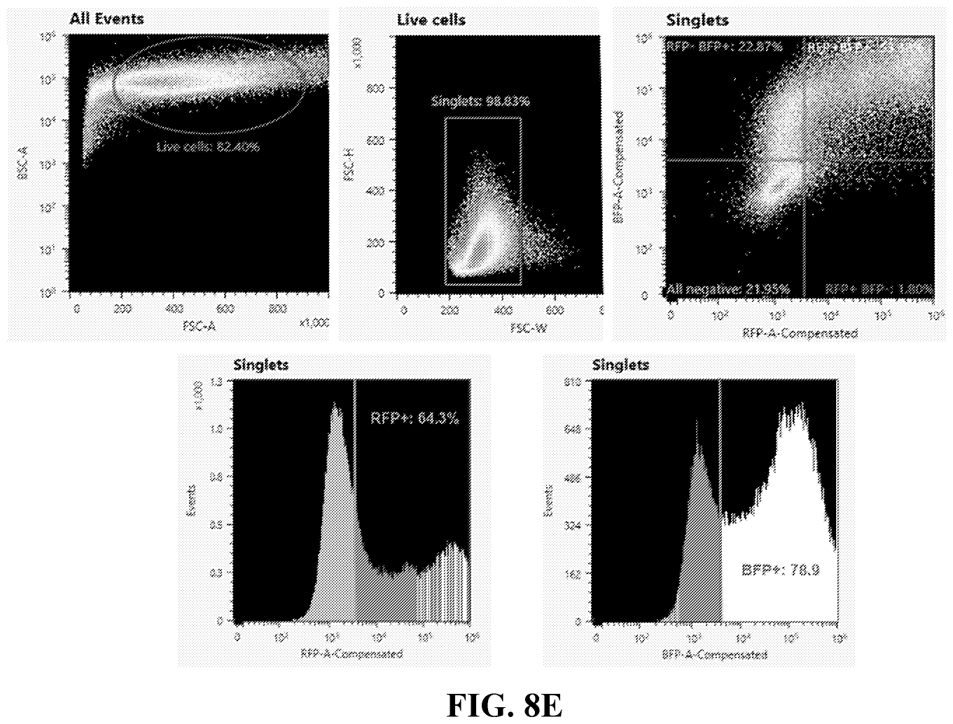

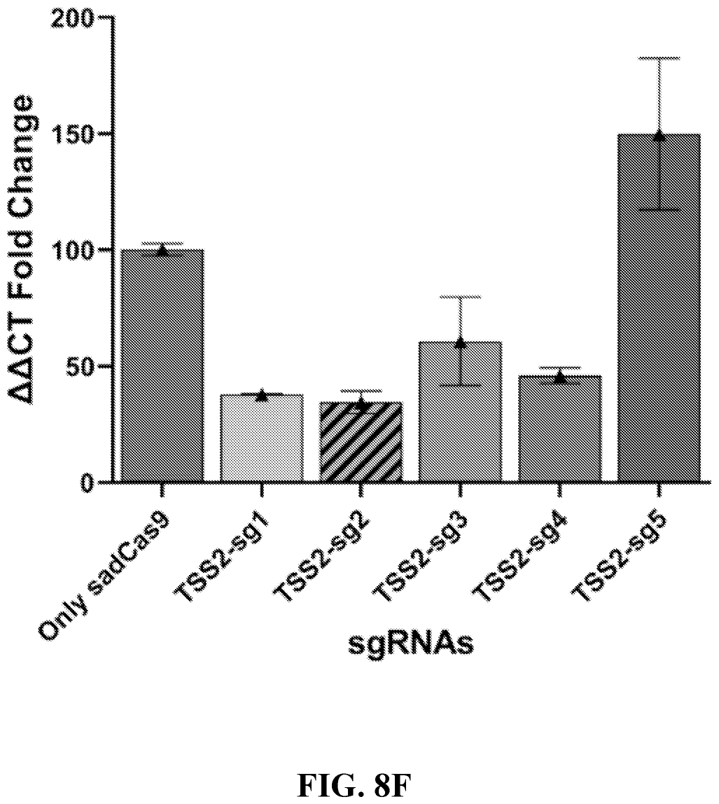

FIGS. 8A-8F illustrate various aspects of screening of sgRNAs targeting transcriptional start site 2 in the human SNCA promoter (TSS2) in the alpha synuclein (SNCA) promoter with HEK293 cells. (A) Schematic representation of vector cassettes for inducible SadCas9 and red fluorescent protein (RFP) and sgRNAs and blue fluorescent protein (BFP) co-expression. (B) UCSC Genome browser view of SNCA promoter region showing five sgRNAs targeted to the transcriptional start site 2 in the human SNCA promoter. (C) Schematic overview of the transient inducible expression of SadCas9 and sgRNA, fluorescent activated cell sorting (FACS) and quantitative PCR amplification with Taqman probes. (D) Representative images of induced expression of SadCas9 with different sgRNAs and negative SadCas9 only control. (E) FACS sorting and gating of RFP and BFP double-positive cells, on average we reached 53.24% double positive cells in HEK293 cultures. (F) Taqman quantitative expression analysis of the SNCA gene, normalized to GAPDH, and compared to SadCas9 only cells.

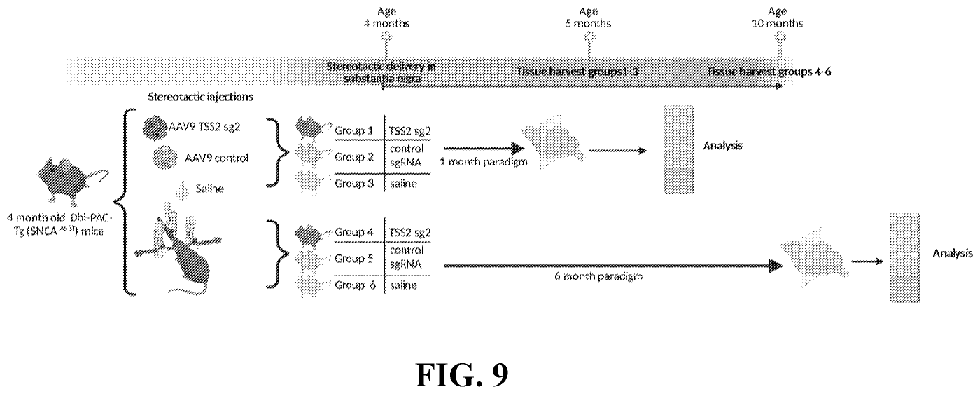

FIG. 9 is an illustration of an embodiment of study design and an embodiment of a timeline of experiments according to the present disclosure. Four-month-old Dbl-PAC-Tg (SNCA^{A53T}) mice were grouped into six experimental Groups: Groups 1 and 4 received AAV9 virus containing the active construct of SadCas9 with TSS2 sgRNA2 (SEQ ID NO:28, alternatively SEQ ID NO:3) stercotactically injected into the substantia nigra: Groups 2 and 5 received AAV9 containing a non-human targeting sgRNA gal4 control; and Groups 3 and 6 received saline controls. Groups 1-3 were euthanized after 1 month and groups 4-6 after 6 months and brains and peripheral organs were harvested and fresh frozen in liquid nitrogen. Coronal sections of 20 um were taken and further stained by in situ hybridization RNAScope or immunofluorescence histology, quantified, and analyzed by Qpath (v 0.4.2).

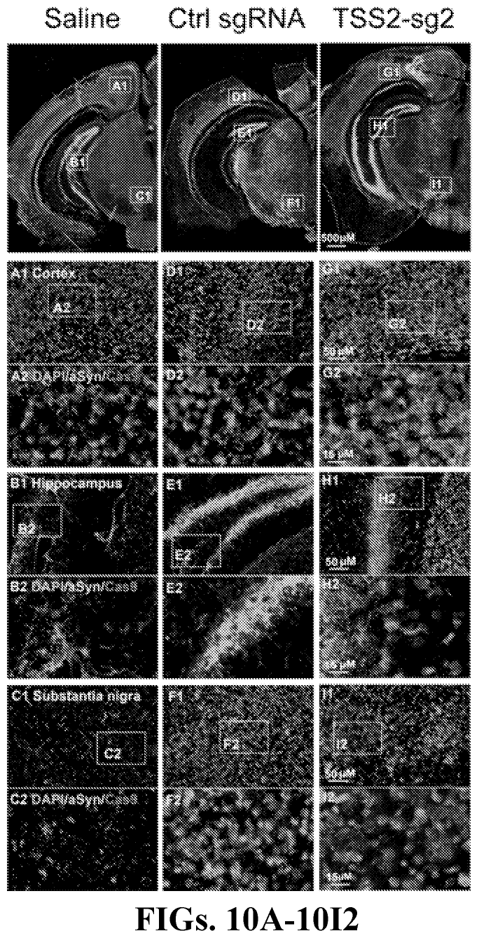

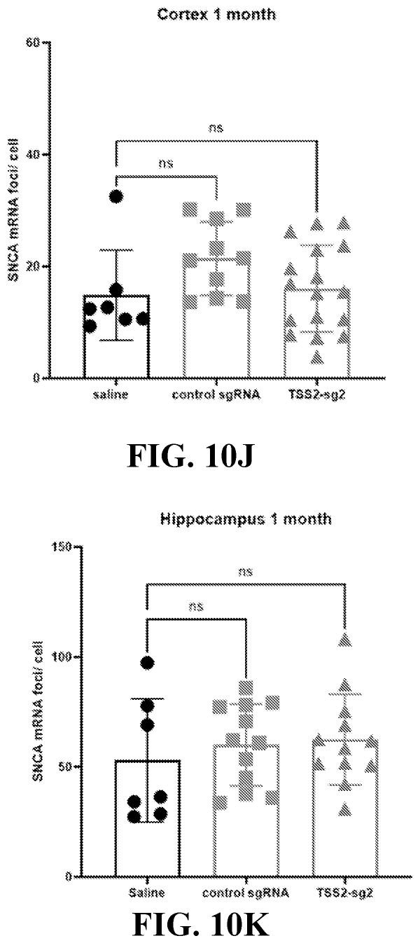

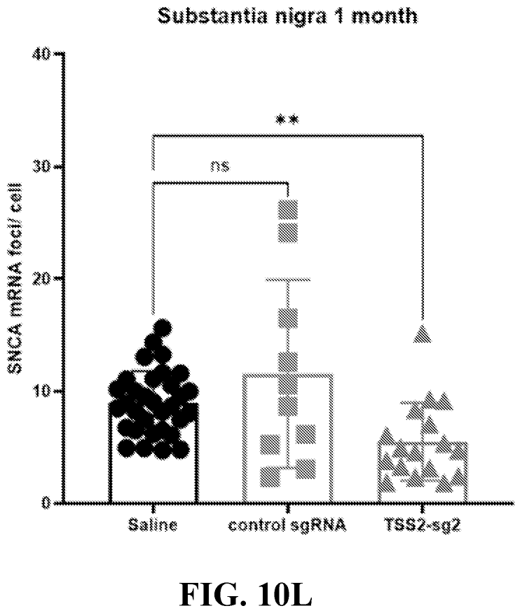

FIGS. 10A-10L show aspects of SNCA mRNA downregulation at 1-month post-surgery according to an embodiment of the present disclosure. SNCA mRNA shows a reduction illustrated as RNAScope foci per cell in the TSS2-sg2 group (FIGS. 10I1 and I2; FIG. 10L) for the substantia nigra. SNCA mRNA signals are unchanged in the hippocampal (FIGS. H1-10H2; FIG. 10K) and cortical areas (FIGS. 10G1-10G2: FIG. 10J). RNA expression was detected using specific in situ hybridization probes (RNAScope) against alpha-synuclein (green) and Cas9 (red), counterstained with DAPI (blue). Panoramic coronal mesencephalic section of brains one-month post-surgery are shown as saline control (top left panel), control (ctrl) sgRNA (top middle panel), and active TSS2-sgRNA2 (top right panel) (scale bar=500 μm). In-situ hybridization signal merged images depict SNCA mRNA (aSyn, green), sadCas9 (red), and DAPI (blue). Respective regions of cortex, dentate gyrus of hippocampus, and substantia nigra at higher magnification of the injected side are shown for saline control group (left panel figures starting one down from the top, FIGS. 10A 1/A2, B1/B2, C1/C2), ctrl sgRNA group (middle panel figures starting one down from the top, FIGS. 10D1/D2, E1/E2, F1/F2), and TSS2-sg2 group (right panel figures starting one down from the top, FIGS. 10G1/G2, H1/H2, I1/I2). Scale bars are indicated at 50 μm (FIGS. 10A1, B1, C1, D1, E1, F1, H1, and I1) and 15 μm (FIGS. 10A2, B2, C2, D2, E2, F2, G2, H2, I2). FIGS. 10K-L are bar graphs quantifying the SNCA mRNA expression signal between the three treatment groups (saline, control sgRNA, TSS2-sg2) according to an embodiment of the present disclosure. Results are shown as ns=non-[statistically] significant, (*) p<0.05, (**) p<0.01, or (***) p<0.001.

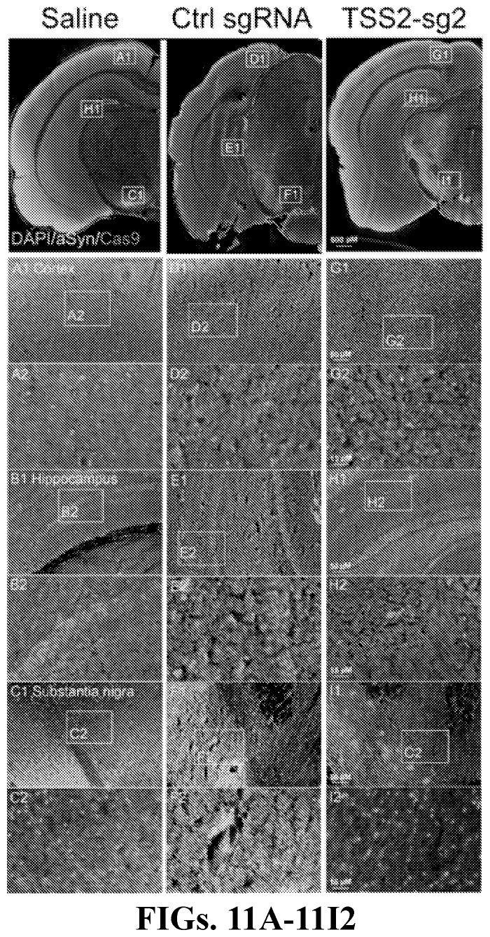

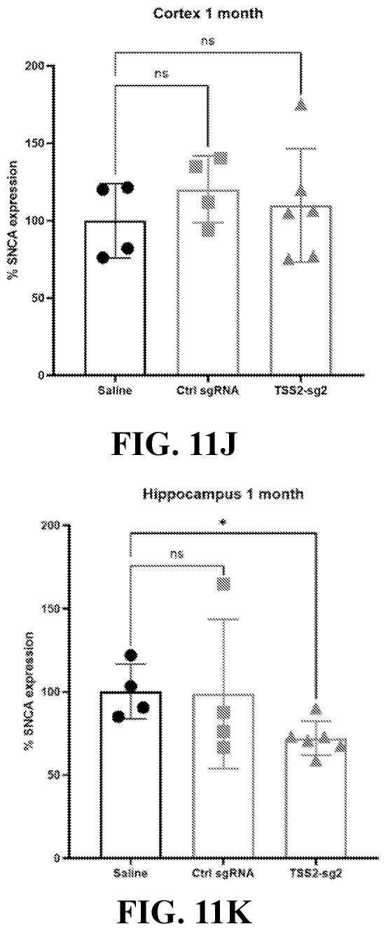

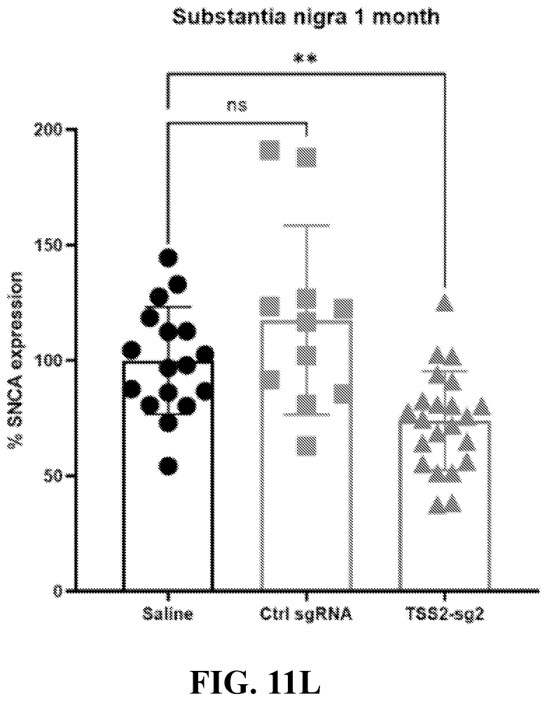

FIGS. 11A-11L show aspects of SadCas9-mediated alpha-synuclein protein downregulation at 1 month post stereotactic surgery in the substantia nigra according to an embodiment of the present disclosure. Alpha-synuclein immunostaining shows a reduction of signal intensity in the TSS2-sg2 group (right panel) for the substantia nigra and hippocampus (FIGS. 11G1-G2, H1-I1: FIG. 11K, FIG. 11L), whereas alpha-synuclein signals are unchanged in the cortical area (FIGS. 11G1-G2; FIG. 11J). Panoramic coronal mesencephalic section of brains one-month post-surgery are shown as saline control (top left panel), control (ctrl) sgRNA (top middle panel), and active TSS2-sgRNA2 (top right panel) (scale bar=500 μm). Immunofluorescence merged images depict alpha-synuclein (aSyn, green), sadCas9 (red), and DAPI (blue). Respective regions of cortex, dentate gyrus of hippocampus, and substantia nigra at higher magnification of the injected side are shown for saline control group (left panel figures starting one down from the top, FIGS. 11A1/A2, B1/B2, C1/C2), ctrl sgRNA group (middle panel figures starting one down from the top, FIGS. 11D1/D2, E1/E2, F1/F2), and TSS2-sg2 group (right panel figures starting one down from the top, FIGS. 11G1/G2, H1/H2, I1/I2). Scale bars are indicated at 50 μm (FIGS. 11A1, B1, C1, D1, E1, F1, H1, and I1) and 15 μm (FIGS. 11A2, B2, C2, D2, E2, F2, G2, H2, I2). FIGS. 11K-L depicts bar graphs quantifying the a-syn expression signal between the three treatment groups (saline, control sgRNA, TSS2-sg2) according to an embodiment of the present disclosure. Results are shown as ns=non-[statistically]significant, (*) p<0.05, (**) p<0.01, or (***) p<0.001.

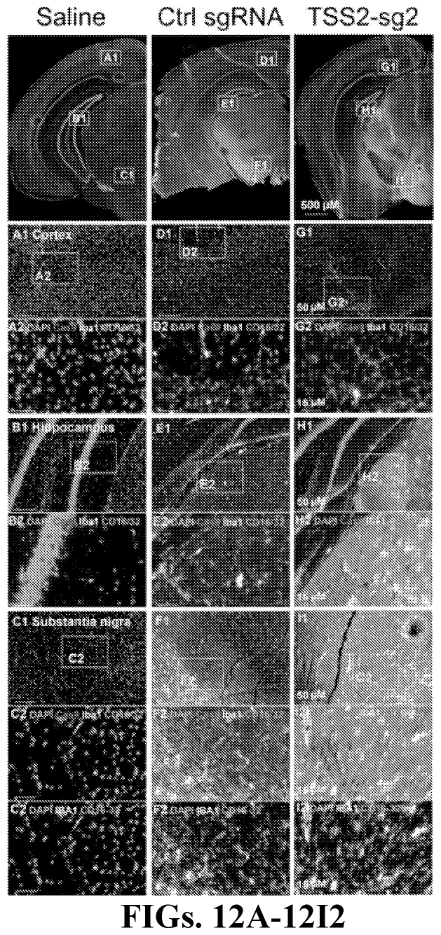

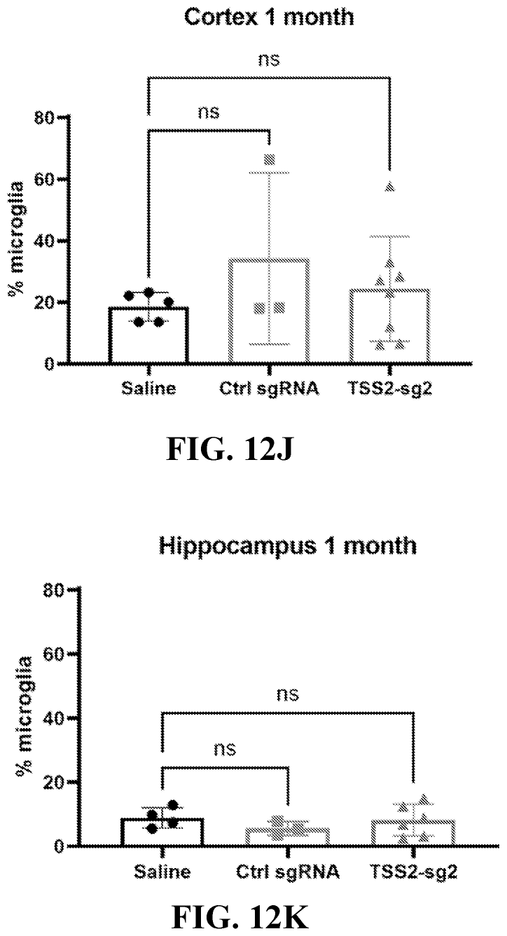

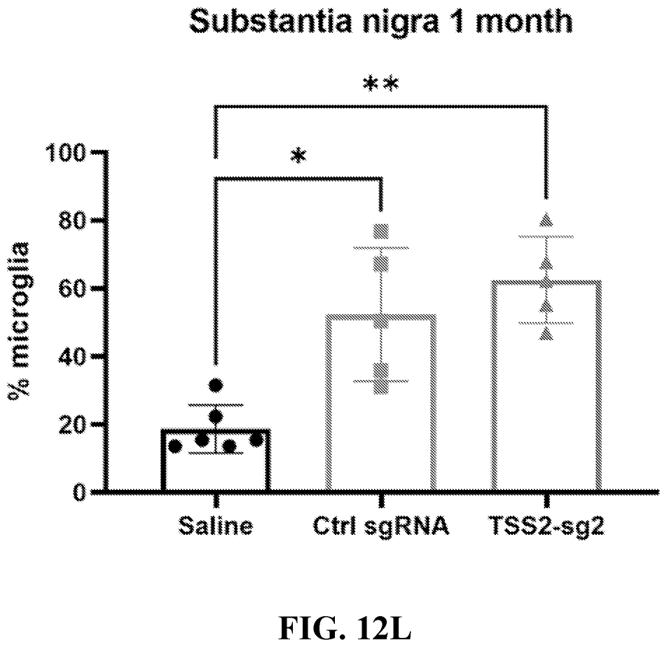

FIGS. 12A-12L show aspects of microglia activation at 1 month post stereotactic surgery in the substantia nigra according to an embodiment of the present disclosure. Ibal immunoreactivity is increased in TSS2-sg2 and ctrl sgRNA group (right and middle panels. FIGS. 12F1-F2, FIGS. 12I1-I2; FIG. 12L) for the substantia nigra, but not in the hippocampus or cortical region (FIGS. 12E1-E2, FIGS. 12D1-12D2; FIG. 12K). Panoramic coronal mesencephalic section of brains one-month post-surgery are shown as saline control (top left panel), control (ctrl) sgRNA (top middle panel), and active TSS2-sgRNA2 (top right panel) (scale bar=500 μm). Immunofluorescence merged images depict sadCas9 (red). Ibal (white), Cd16/32 (green) and DAPI (blue). Respective regions of cortex, dentate gyrus of hippocampus, and substantia nigra at higher magnification of the injected side are shown for saline control group (left panel figures starting one down from the top, FIGS. 12A1/A2, B1/B2, C1/C2), ctrl sgRNA group (middle panel figures starting one down from the top, FIGS. 12D1/D2, E1/E2, F1/F2), and TSS2-sg2 group (right panel figures starting one down from the top, FIGS. 12G1/G2, H1/H2, I1/I2). Scale bars are indicated at 50 μm (FIGS. 12A1, B1, C1, D1, E1, F1, H1, and I1) and 15 μm (FIGS. 12A2, B2, C2, D2, E2, F2, G2, H2, I2). FIGS. 12K-L depicts bar graphs quantifying the % Ibal microglia signal between the three treatment groups (saline, control sgRNA, TSS2-sg2) according to an embodiment of the present disclosure. Results are shown as ns=non-significant. (*) p<0.05, (**) p<0.01, or (***) p<0.001.

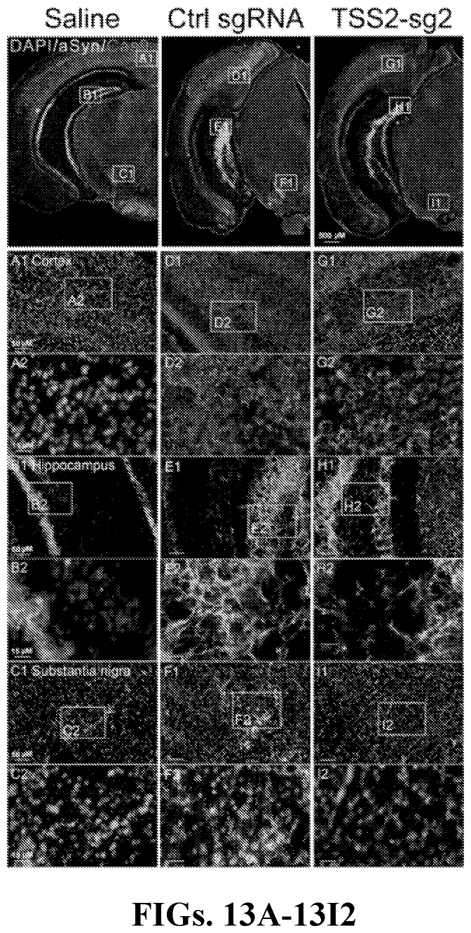

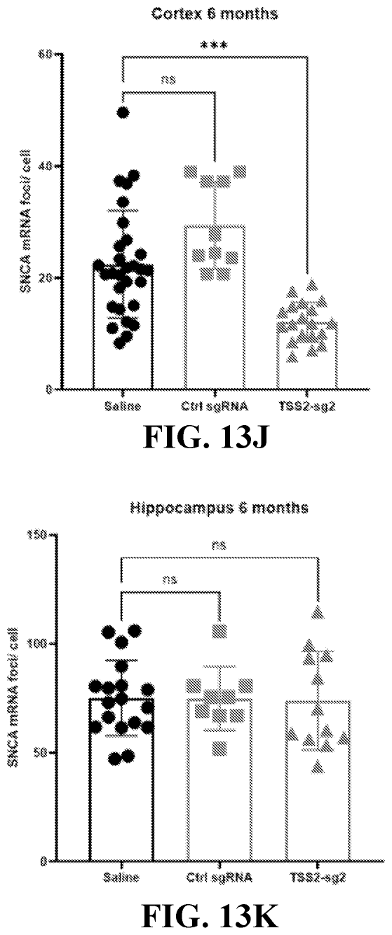

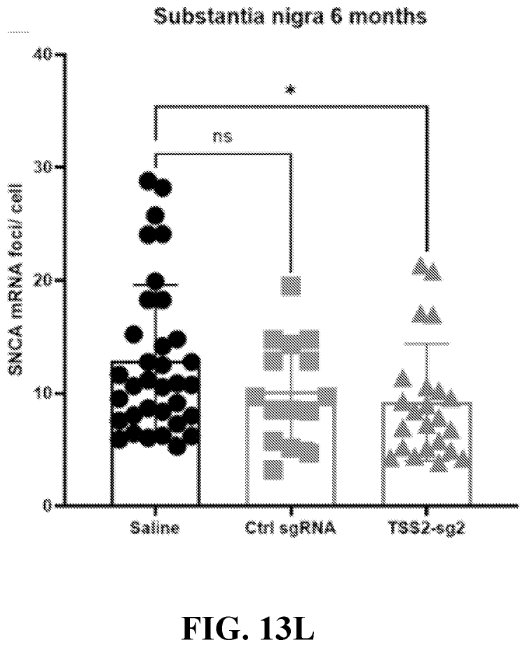

FIGS. 13A-13L show aspects of SNCA mRNA downregulation at 6-months post-surgery according to an embodiment of the present disclosure. SNCA mRNA shows a reduction illustrated as RNAScope foci per cell in the TSS2-sg2 group (top right panel) for the substantia nigra and cortex. SNCA mRNA signals are unchanged in the hippocampal area. RNA expression is detected using specific in situ hybridization probes (RNAScope) against alpha-synuclein (green) and Cas9 (red) counterstained with DAPI (blue). Panoramic coronal mesencephalic section of brains 6 months post-surgery are shown as saline control (top left panel), control (ctrl) sgRNA (top middle panel), and active TSS2-sgRNA2 (top right panel) (scale bar=500 μm). In situ hybridization signal merged images depict SNCA mRNA (aSyn, green), sadCas9 (red), and DAPI (blue). Respective regions of cortex, dentate gyrus of hippocampus, and substantia nigra at higher magnification of the injected side are shown for saline control group (left panel figures starting one down from the top, FIGS. 13A1/A2, B1/B2, C1/C2), ctrl sgRNA group (middle panel figures starting one down from the top, FIGS. 13D1/D2. E1/E2, F1/F2), and TSS2-sg2 group (right panel figures starting one down from the top. FIGS. 13G1/G2, H1/H2, I1/I2). Scale bars are indicated at 50 μm (FIGS. 13A1, B1, C1, D1, E1, F1, H1, and I1) and 15 μm (FIGS. 13A2, B2, C2, D2, E2, F2, G2, H2, I2). FIGS. 13K-L depicts bar graphs quantifying the SNCA mRNA expression signal between the three treatment groups (saline, control sgRNA, TSS2-sg2) according to an embodiment of the present disclosure. Results are shown as ns=non-[statistically] significant, (*) p<0.05, (**) p<0.01, or (***) p<0.001.

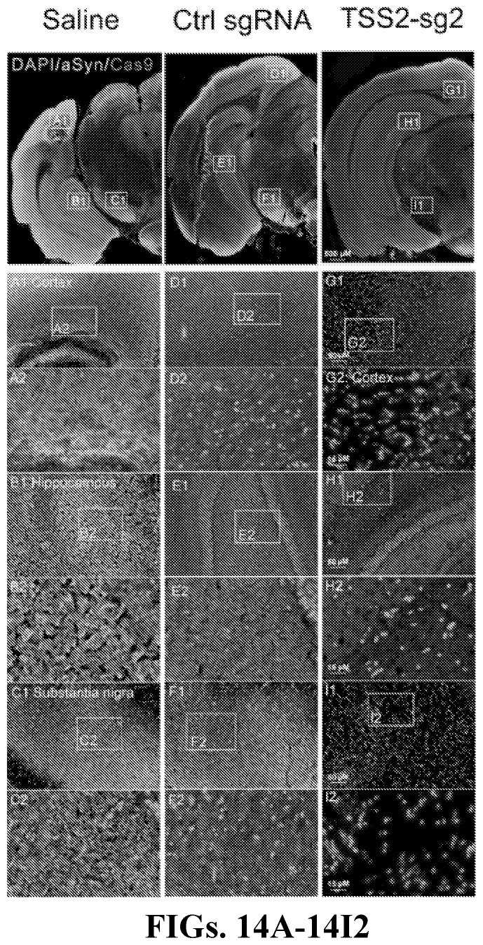

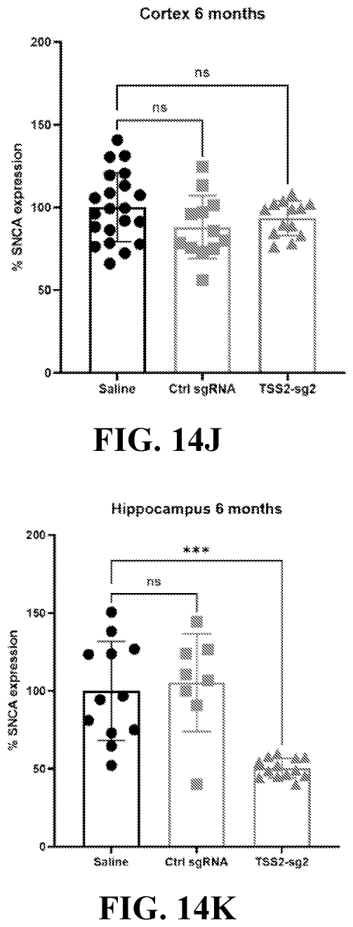

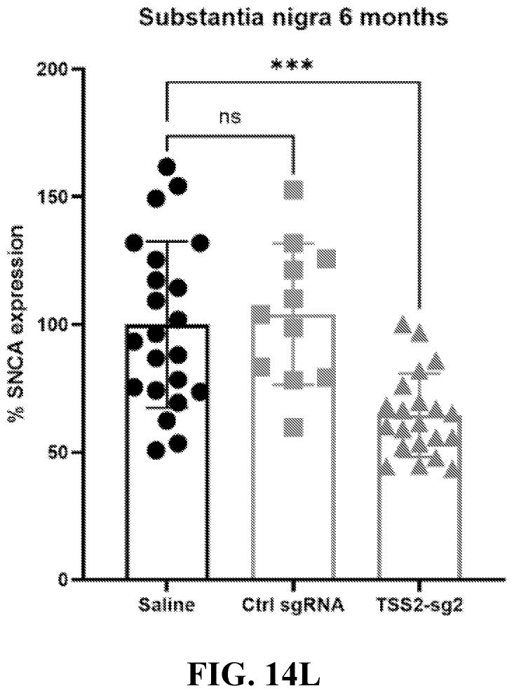

FIGS. 14A-14L show aspects of SadCas9-mediated alpha-synuclein protein downregulation 6 months post stereotactic surgery in substantia nigra and hippocampus according to an embodiment of the present disclosure. Alpha-synuclein immunostaining shows a reduction of signal intensity in the TSS2-sg2 group for the substantia nigra and hippocampus (FIGS. 14I-I2, FIGS. 14H1-H2, respectively), whereas alpha-synuclein signals are unchanged in the cortical area (FIGS. 14G1-G2). Panoramic coronal mesencephalic section of brains 6 months post-surgery are shown as saline control (top left panel), control (ctrl) sgRNA (top middle panel), and active TSS2-sgRNA2 (top right panel) (scale bar=500 μm). Immunofluorescence merged images depict alpha-synuclein (aSyn, green), sadCas9 (red), and DAPI (blue). Respective regions of cortex, dentate gyrus of hippocampus, and substantia nigra at higher magnification of the injected side are shown for saline control group (left panel figures starting one down from the top, FIGS. 14A1/A2, B1/B2, C1/C2), ctrl sgRNA group (middle panel figures starting one down from the top, FIGS. 14D1/D2, E1/E2, F1/F2), and TSS2-sg2 group (right panel figures starting one down from the top. FIGS. 14G1/G2, H1/H2, I1/I2). Scale bars are indicated at 50 μm (FIGS. 14A1, B1, C1, D1, E1, F1, H1, and I1) and 15 μm (FIGS. 14A2, B2, C2, D2, E2, F2, G2, H2, I2). FIGS. 14K-L depicts bar graphs quantifying the a-syn expression signal between the three treatment groups (saline, control sgRNA, TSS2-sg2) according to an embodiment of the present disclosure Results are shown as ns=non-[statistically] significant, (*) p<0.05, (**) p<0.01, or (***) p<0.001.

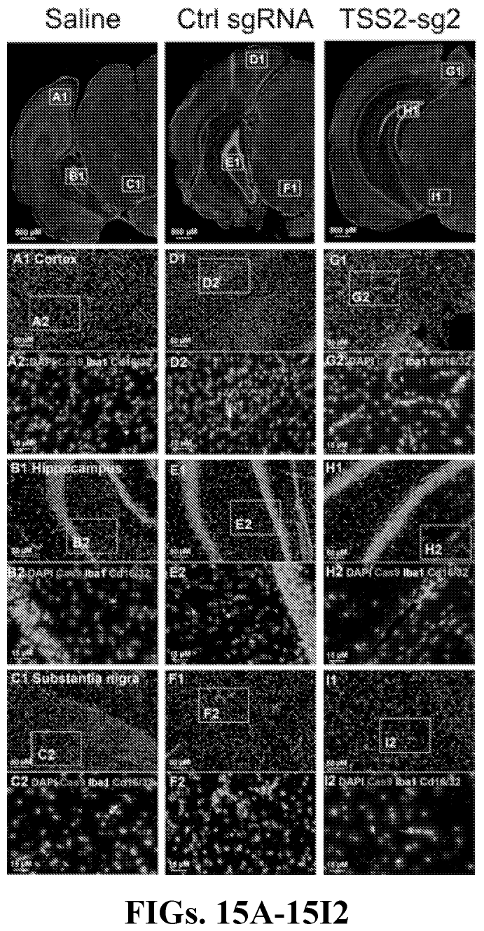

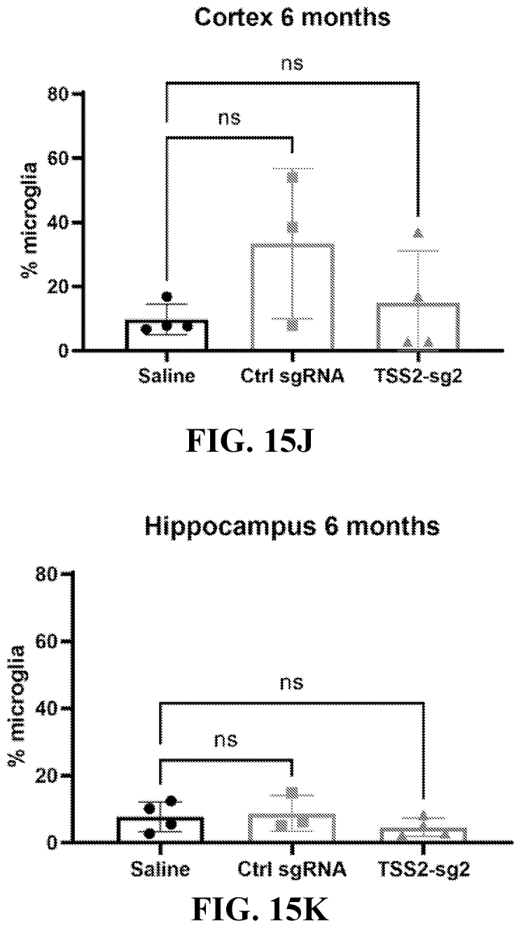

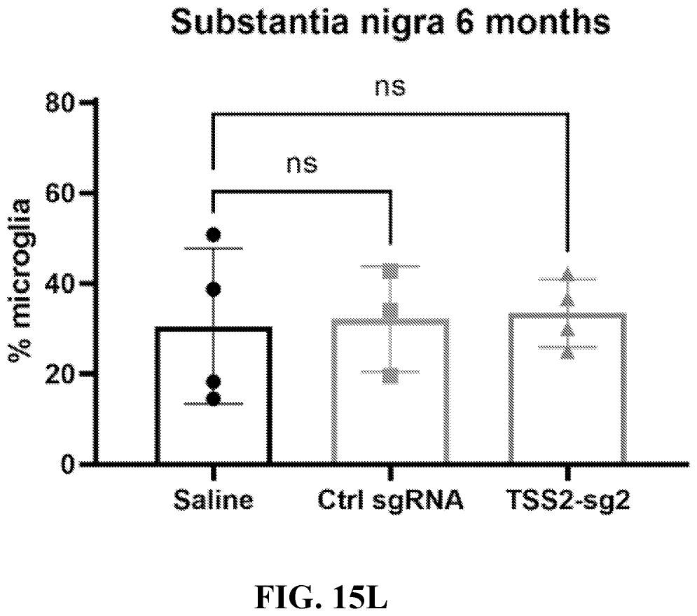

FIGS. 15A-15L show aspects of microglial activation 6-months post stereotactic surgery in the substantia nigra according to an embodiment of the present disclosure. Ibal immunoreactivity is unchanged between the three treatment groups (saline, control sgRNA, TSS2-sg2). Panoramic coronal mesencephalic section of brains 6 months post-surgery are shown as saline control (top left panel), control (ctrl) sgRNA (top middle panel), and active TSS2-sgRNA2 (top right panel) (scale bar=500 μm). Immunofluorescence merged images depict sadCas9 (red), Ibal (white), Cd16/32 (green) and DAPI (blue). Respective regions of cortex, dentate gyrus of hippocampus, and substantia nigra at higher magnification of the injected side are shown for saline control group (left panel figures starting one down from the top, FIGS. 15A1/A2, B1/B2, C1/C2), ctrl sgRNA group (middle panel figures starting one down from the top, FIGS. 15D1/D2, E1/E2, F1/F2), and TSS2-sg2 group (right panel figures starting one down from the top, FIGS. 15G1/G2, H1/H2, I1/I2). Scale bars are indicated at 50 μm (FIGS. 15A1, B1, C1, D1, E1, F1, H1, and I1) and 15 μm (FIGS. 15A2, B2, C2, D2, E2, F2, G2, H2, I2). FIGS. 15K-L depicts bar graphs quantifying the % Ibal microglia signal between the three treatment groups (saline, control sgRNA, TSS2-sg2) according to an embodiment of the present disclosure. Results are shown as ns=non-[statistically] significant, (*) p<0.05, (**) p<0.01, or (***) p<0.001.

### Exemplary Embodiments

Exemplary embodiments provided in accordance with the presently disclosed subject matter include, but are not limited to, the claims and the following embodiments:

Embodiment 1. A recombinant DNA molecule comprising a nucleic acid sequence that encodes a fusion protein comprising a clustered regularly interspaced short palindromic repeats (CRISPR)-associated nuclease and a transcriptional repressor.

Embodiment 2. The recombinant DNA molecule of embodiment 1, further comprising a nucleic acid sequence that encodes a guide RNA that is complementary to a sequence in an alpha-synuclein gene or a gene regulatory region thereof.

Embodiment 3. The recombinant DNA molecule of embodiment 2, wherein the alpha-synuclein gene is a human or non-human primate alpha-synuclein gene.

Embodiment 4. The recombinant DNA molecule of embodiment 2 or 3, wherein the nucleic acid sequence that encodes a guide RNA comprises a sequence that is at least 90% identical to SEQ ID NO: 1, 2, 3, 24, 25, 26, 27, or 28.

Embodiment 5. The recombinant DNA molecule of any one of embodiments 1 to 4, wherein the CRISPR-associated nuclease is Cas9.

Embodiment 6. The recombinant DNA molecule of embodiment 5, wherein the Cas9 is Staphylococcus aureus Cas9.

Embodiment 7. The recombinant DNA molecule of any one of embodiments 1 to 6, wherein the CRISPR-associated nuclease comprises one or more mutations relative to the wild-type CRISPR-associated nuclease.

Embodiment 8. The recombinant DNA molecule of embodiment 7, wherein at least one of the one or more mutations results in a reduction in CRISPR-associated nuclease catalytic activity.

Embodiment 9. The recombinant DNA molecule of embodiment 8, wherein the one or more mutations results in a catalytically inactive CRISPR-associated nuclease.

Embodiment 10. The recombinant DNA molecule of any one of embodiments 7 to 9, wherein the CRISPR-associated nuclease is S. aureus Cas9 and wherein the S. aureus Cas9 comprises a D10A mutation and/or a N580A mutation.

Embodiment 11. The recombinant DNA molecule of any one of embodiments 1 to 10, wherein the transcriptional repressor is a Krüppel associated box (KRAB) domain.

Embodiment 12. The recombinant DNA molecule of embodiment 11, wherein the fusion protein comprises at least 2 KRAB domains.

Embodiment 13. The recombinant DNA molecule of any one of embodiments 1 to 12, wherein the fusion protein further comprises a nuclear localization signal (NLS).

Embodiment 14. A DNA construct comprising the recombinant DNA molecule of any one of embodiments 1 to 13 operably linked to a constitutive or inducible promoter.

Embodiment 15. The DNA construct of embodiment 14, wherein the promoter is a human methyl CpG binding protein 2 (MECP2) promoter, a human synapsin promoter (SYN1), or a human phosphoglycerate kinase (PGK) promoter.

Embodiment 16. The DNA construct of embodiment 14, wherein the DNA construct comprises two promoters: i) a first promoter operably linked to the nucleic acid sequence that encodes a fusion protein comprising a CRISPR-associated nuclease and a transcriptional repressor; and ii) a second promoter operably linked to the nucleic acid sequence that encodes a guide RNA that is complementary to a sequence in an alpha-synuclein gene or a gene regulatory region thereof.

Embodiment 17. The DNA construct of embodiment 16, wherein the first promoter is a human MECP2 promoter, a human synapsin promoter, or a human PGK promoter.

Embodiment 18. The DNA construct of embodiment 16 or 17, wherein the second promoter is a human U6 promoter.

Embodiment 19. A vector comprising the DNA construct of any one of embodiments 14 to 18.

Embodiment 20. The vector of embodiment 19, wherein the vector is a viral vector.

Embodiment 21. The vector of embodiment 20, wherein the vector is an adeno-associated virus vector.

Embodiment 22. The vector of embodiment 21, wherein the adeno-associated virus is AAV9.

Embodiment 23. The vector of any one of embodiments 19 to 22, wherein the vector is up to 4800 bp in length.

Embodiment 24. The vector of embodiment 23, wherein the vector is up to 5100 bp in length.

Embodiment 25. The vector of any one of embodiments 19 to 24, wherein the DNA construct comprises a sequence that is at least 90% identical to SEQ ID NO:20 or 21.

Embodiment 26. An isolated virus comprising the vector of any one of embodiments 19 to 25.

Embodiment 27. The isolated virus of embodiment 26, wherein the virus is an adeno-associated virus.

Embodiment 28. The isolated virus of embodiment 27, wherein the adeno-associated virus is AAV9.

Embodiment 29. A composition comprising: i) the recombinant DNA molecule of any one of embodiments 1 to 13, the DNA construct of any one of embodiments 14 to 18, the vector of any one of embodiments 19 to 25, or the isolated virus of any one of embodiments 26 to 28; and ii) a pharmaceutically acceptable carrier.

Embodiment 30. A method of treating a subject with Parkinson's disease, the method comprising administering to the subject a therapeutically effective amount of the composition of embodiment 29.

Embodiment 31. The method of embodiment 30, wherein the composition is administered intrathecally.

Embodiment 32. The method of embodiment 30 or 31, wherein the composition is administered into the cisterna magna.

Embodiment 33. The method of any one of embodiments 30 to 32, wherein the composition is administered into cerebrospinal fluid.

Embodiment 34. The method of any one of embodiments 30 to 33, wherein the administration of the composition results in a decreased amount of alpha-synuclein protein expression in the subject relative to the amount of alpha-synuclein protein expression in the subject prior to administration of the composition.

SEQUENCES ACCORDING TO THE PRESENT DISCLOSURE
(5′→3′ unless otherwise noted)

SEQ ID NO: 1-sgRNA3
ACTTCTGATTCTCGTTGCCCT
SEQ ID NO: 2-sgRNA4
CACCCTCGTGAGCGGAGAACT
SEQ ID NO: 3-sgRNA6 (also referred to as “RNA6”)
GTGGCCATTCGACGACAGGTTA
SEQ ID NO: 4-NLS of the SV40 virus large T-antigen
PKKKRKV
SEQ ID NO: 5-the NLS from nucleoplasmin (c.g. the nucleoplasmin bipartite NLS)
KRPAATKKAGQAKKKK
SEQ ID NO: 6-c-myc NLS
PAAKRVKLD
SEQ ID NO: 7-c-myc NLS (alternate)
RQRRNELKRSP
SEQ ID NO: 8-bRNPA1 M9 NLS
NQSSNFGPMKGGNFGGRSSGPYGGGGQYFAKPRNQGGY
SEQ ID NO: 9-IBB domain from importin-alpha
RMRIZFKNKGKDTAELRRRRVEVSVELRKAKKDEQILKRRNV
SEQ ID NO: 10-Portion of the myoma T protein
VSRKRPRP
SEQ ID NO: 11-Portion of the myoma T protein (alternate)
PPKKARED
SEQ ID NO: 12-Portion of human p53
PQPKKKPL
SEQ ID NO: 13-Portion of mouse c-abl IV
SALIKKKKKMAP
SEQ ID NO: 14-Portion of the influenza virus NS1
DRLRR
SEQ ID NO: 15-Portion of the influenza virus NS1 (alternate)
PKQKKRK
SEQ ID NO: 16-Portion of the Hepatitis virus delta antigen
RKLKKKIKKL
SEQ ID NO: 17-Portion of the mouse Mx1 protein
REKKKFLKRR
SEQ ID NO: 18-Portion of the human poly(ADP-ribose) polymerase
KRKGDEVDGVDEVAKKKSKK
SEQ ID NO: 19-Portion of steroid hormone receptor[s] (human) glucocorticoid
RKCLQAGMNLEARKTKK
SEQ ID NO: 20-pAAV9-U6>RNA6-SYN1>SadCas9/2xKRAB: SV40 pA [sgRNA
underlined, RNA6 (SEQ ID NO: 3); RNA6/sa optimized scaffold italicized]
CCTGCAGGCAGCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCATCAC
TAGGGGTTCCTTCTAGAGAGGGCCTATTTCCCATGATTCCTTCATATTTGCATATACGATACAAGGC
TGTTAGAGAGATAATTGGAATTAATTTGACTGTAAACACAAAGATATTAGTACAAAATACGTGAC
GTAGAAAGTAATAATTTCTTGGGTAGTTTGCAGTTTTAAAATTATGTTTTAAAATGGACTATCATAT
GCTTACCGTAACTTGAAAGTATTTCGATTTCTTGGCTTTATATATCTTGTGGAAAGGACGAAACAC
CGTGGCCATTCGACGACAGGTTAGTTATAGTACTCTGGAAACAGAATCTACTATAACAAGGCAAAATGCCG
TGTTTATCTCGTCAACTTGTTGGCGAGATTTTTTTCTGCAGAGGGCCCTGCGTATGAGTGCAAGTGGGT
TTTAGGACCAGGATGAGGCGGGGGGGGGTGCCTACCTGACGACCGACCCCGACCCACTGGACAA
GCACCCAACCCCCATTCCCCAAATTGCGCATCCCCTATCAGAGAGGGGGAGGGGAAACAGGATGC
GGCGAGGCGCGTGCGCACTGCCAGCTTCAGCACCGCGGACAGTGCCTTCGCCCCCGCCTGGCGGC
GCGCGCCACCGCCGCCTCAGCACTGAAGGCGCGCTGACGTCACTCGCCGGTCCCCCGCAAACTCCC
CTTCCCGGCCACCTTGGTCGCGTCCGCGCCGCCGCCGGCCCAGCCGGACCGCACCACGCGAGGCG
CGAGATAGGGGGGCACGGGCGCGACCATCTGCGCTGCGGCGCCGGCGACTCAGCGCTGCCTCAGT
CTGCGGTGGGCAGCGGAGGAGTCGTGTCGTGCCTGAGAGCGCAGGCCACCATGTACCCATACGAT
GTTCCAGATTACGCTGCCCCAAAGAAGAAGCGGAAGGTCGGTATCCACGGAGTCCCAGCAGCCAA
GCGGAACTACATCCTGGGCCTGGCCATCGGCATCACCAGCGTGGGCTACGGCATCATCGACTACG
AGACACGGGACGTGATCGATGCCGGCGTGCGGCTGTTCAAAGAGGCCAACGTGGAAAACAACGA
GGGCAGGCGGAGCAAGAGAGGCGCCAGAAGGCTGAAGCGGCGGAGGCGGCATAGAATCCAGAG
AGTGAAGAAGCTGCTGTTCGACTACAACCTGCTGACCGACCACAGCGAGCTGAGCGGCATCAACC
CCTACGAGGCCAGAGTGAAGGGCCTGAGCCAGAAGCTGAGCGAGGAAGAGTTCTCTGCCGCCCTG
CTGCACCTGGCCAAGAGAAGAGGCGTGCACAACGTGAACGAGGTGGAAGAGGACACCGGCAACG
AGCTGTCCACCAAAGAGCAGATCAGCCGGAACAGCAAGGCCCTGGAAGAGAAATACGTGGCCGA
ACTGCAGCTGGAACGGCTGAAGAAAGACGGCGAAGTGCGGGGCAGCATCAACAGATTCAAGACC
AGCGACTACGTGAAAGAAGCCAAACAGCTGCTGAAGGTGCAGAAGGCCTACCACCAGCTGGACC
AGAGCTTCATCGACACCTACATCGACCTGCTGGAAACCCGGCGGACCTACTATGAGGGACCTGGC
GAGGGCAGCCCCTTCGGCTGGAAGGACATCAAAGAATGGTACGAGATGCTGATGGGCCACTGCAC
CTACTTCCCCGAGGAACTGCGGAGCGTGAAGTACGCCTACAACGCCGACCTGTACAACGCCCTGA
ACGACCTGAACAATCTCGTGATCACCAGGGACGAGAACGAGAAGCTGGAATATTACGAGAAGTTC
CAGATCATCGAGAACGTGTTCAAGCAGAAGAAGAAGCCCACCCTGAAGCAGATCGCCAAAGAAA
TCCTCGTGAACGAAGAGGATATTAAGGGCTACAGAGTGACCAGCACCGGCAAGCCCGAGTTCACC
AACCTGAAGGTGTACCACGACATCAAGGACATTACCGCCCGGAAAGAGATTATTGAGAACGCCGA
GCTGCTGGATCAGATTGCCAAGATCCTGACCATCTACCAGAGCAGCGAGGACATCCAGGAAGAAC
TGACCAATCTGAACTCCGAGCTGACCCAGGAAGAGATCGAGCAGATCTCTAATCTGAAGGGCTAT
ACCGGCACCCACAACCTGAGCCTGAAGGCCATCAACCTGATCCTGGACGAGCTGTGGCACACCAA
CGACAACCAGATCGCTATCTTCAACCGGCTGAAGCTGGTGCCCAAGAAGGTGGACCTGTCCCAGC
AGAAAGAGATCCCCACCACCCTGGTGGACGACTTCATCCTGAGCCCCGTCGTGAAGAGAAGCTTC
ATCCAGAGCATCAAAGTGATCAACGCCATCATCAAGAAGTACGGCCTGCCCAACGACATCATTAT
CGAGCTGGCCCGCGAGAAGAACTCCAAGGACGCCCAGAAAATGATCAACGAGATGCAGAAGCGG
AACCGGCAGACCAACGAGCGGATCGAGGAAATCATCCGGACCACCGGCAAAGAGAACGCCAAGT
ACCTGATCGAGAAGATCAAGCTGCACGACATGCAGGAAGGCAAGTGCCTGTACAGCCTGGAAGCC
ATCCCTCTGGAAGATCTGCTGAACAACCCCTTCAACTATGAGGTGGACCACATCATCCCCAGAAGC
GTGTCCTTCGACAACAGCTTCAACAACAAGGTGCTCGTGAAGCAGGAAGAAGCCAGCAAGAAGGG
CAACCGGACCCCATTCCAGTACCTGAGCAGCAGCGACAGCAAGATCAGCTACGAAACCTTCAAGA
AGCACATCCTGAATCTGGCCAAGGGCAAGGGCAGAATCAGCAAGACCAAGAAAGAGTATCTGCTG
GAAGAACGGGACATCAACAGGTTCTCCGTGCAGAAAGACTTCATCAACCGGAACCTGGTGGATAC
CAGATACGCCACCAGAGGCCTGATGAACCTGCTGCGGAGCTACTTCAGAGTGAACAACCTGGACG
TGAAAGTGAAGTCCATCAATGGCGGCTTCACCAGCTTTCTGCGGCGGAAGTGGAAGTTTAAGAAA
GAGCGGAACAAGGGGTACAAGCACCACGCCGAGGACGCCCTGATCATTGCCAACGCCGATTTCAT
CTTCAAAGAGTGGAAGAAACTGGACAAGGCCAAAAAAGTGATGGAAAACCAGATGTTCGAGGAA
AAGCAGGCCGAGAGCATGCCCGAGATCGAAACCGAGCAGGAGTACAAAGAGATCTTCATCACCCC
CCACCAGATCAAGCACATTAAGGACTTCAAGGACTACAAGTACAGCCACCGGGTGGACAAGAAGC
CTAATAGAGAGCTGATTAACGACACCCTGTACTCCACCCGGAAGGACGACAAGGGCAACACCCTG
ATCGTGAACAATCTGAACGGCCTGTACGACAAGGACAATGACAAGCTGAAAAAGCTGATCAACAA
GAGCCCCGAAAAGCTGCTGATGTACCACCACGACCCCCAGACCTACCAGAAACTGAAGCTGATTA
TGGAACAGTACGGCGACGAGAAGAATCCCCTGTACAAGTACTACGAGGAAACCGGGAACTACCTG
ACCAAGTACTCCAAAAAGGACAACGGCCCCGTGATCAAGAAGATTAAGTATTACGGCAACAAACT
GAACGCCCATCTGGACATCACCGACGACTACCCCAACAGCAGAAACAAGGTCGTGAAGCTGTCCC
TGAAGCCCTACAGATTCGACGTGTACCTGGACAATGGCGTGTACAAGTTCGTGACCGTGAAGAAT
CTGGATGTGATCAAAAAAGAAAACTACTACGAAGTGAATAGCAAGTGCTATGAGGAAGCTAAGA
AGCTGAAGAAGATCAGCAACCAGGCCGAGTTTATCGCCTCCTTCTACAACAACGATCTGATCAAG
ATCAACGGCGAGCTGTATAGAGTGATCGGCGTGAACAACGACCTGCTGAACCGGATCGAAGTGAA
CATGATCGACATCACCTACCGCGAGTACCTGGAAAACATGAACGACAAGAGGCCCCCCAGGATCA
TTAAGACAATCGCCTCCAAGACCCAGAGCATTAAGAAGTACAGCACAGACATTCTGGGCAACCTG
TATGAAGTGAAATCTAAGAAGCACCCTCAGATCATCAAAAAGGGCAAAAGGCCGGCGGCCACGA
AAAAGGCCGGCCAGGCAAAAAAGAAAAAGCATATGACTAGTGGCGGTGGCGGAGGGATGGATGC
TAAGTCACTAACTGCCTGGTCCCGGACACTGGTGACCTTCAAGGATGTATTTGTGGACTTCACCAG
GGAGGAGTGGAAGCTGCTGGACACTGCTCAGCAGATCGTGTACAGAAATGTGATGCTGGAGAACT
ATAAGAACCTGGTTTCCTTGGGTTATCAGCTTACTAAGCCAGATGTGATCCTCCGGTTGGAGAAGG
GAGAAGAGCCCACTAGTGGCGGTGGCGGAGGGATGGATGCTAAGTCACTAACTGCCTGGTCCCGG
ACACTGGTGACCTTCAAGGATGTATTTGTGGACTTCACCAGGGAGGAGTGGAAGCTGCTGGACAC
TGCTCAGCAGATCGTGTACAGAAATGTGATGCTGGAGAACTATAAGAACCTGGTTTCCTTGGGTTA
TCAGCTTACTAAGCCAGATGTGATCCTCCGGTTGGAGAAGGGAGAAGAGCCCTAGCTAGCTTTATT
TGTGAAATTTGTGATGCTATTGCTTTATTTGTAACCATTATAAGCTGCAATAAACAAGTTAACAAC
AACAATTGCATTCATTTTATGTTTCAGGTTCAGGGGGAGATGTGGGAGGTTTTTTAAAGCATGCTG
GGGAGGGCCGCAGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTCAGTGAGCGAGC
GAGCGCGCAGCTGCCTGCAGGGGCGCCTGATGCGGTATTTTCTCCTTACGCATCTGTGCGGTATTT
CACACCGCATACGTCAAAGCAACCATAGTACGCGCCCTGTAGCGGCGCATTAAGCGCGGCGGGGG
TGGTGGTTACGCGCAGCGTGACCGCTACACTTGCCAGCGCCTTAGCGCCCGCTCCTTTCGCTTTCTT
CCCTTCCTTTCTCGCCACGTTCGCCGGCTTTCCCCGTCAAGCTCTAAATCGGGGGCTCCCTTTAGGG
TTCCGATTTAGTGCTTTACGGCACCTCGACCCCAAAAAACTTGATTTGGGTGATGGTTCACGTAGT
GGGCCATCGCCCTGATAGACGGTTTTTCGCCCTTTGACGTTGGAGTCCACGTTCTTTAATAGTGGAC
TCTTGTTCCAAACTGGAACAACACTCAACTCTATCTCGGGCTATTCTTTTGATTTATAAGGGATTTT
GCCGATTTCGGTCTATTGGTTAAAAAATGAGCTGATTTAACAAAAATTTAACGCGAATTTTAACAA
AATATTAACGTTTACAATTTTATGGTGCACTCTCAGTACAATCTGCTCTGATGCCGCATAGTTAAGC
CAGCCCCGACACCCGCCAACACCCGCTGACGCGCCCTGACGGGCTTGTCTGCTCCCGGCATCCGCT
TACAGACAAGCTGTGACCGTCTCCGGGAGCTGCATGTGTCAGAGGTTTTCACCGTCATCACCGAAA
CGCGCGAGACGAAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTGGGTGCACGAG
TGGGTTACATCGAACTGGATCTCAACAGCGGTAAGATCCTTGAGAGTTTTCGCCCCGAAGAACGTT
TTCCAATGATGAGCACTTTTAAAGTTCTGCTATGTGGCGCGGTATTATCCCGTATTGACGCCGGGC
AAGAGCAACTCGGTCGCCGCATACACTATTCTCAGAATGACTTGGTTGAGTACTCACCAGTCACAG
AAAAGCATCTTACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACCGCTTTTTTGCAC
AACATGGGGGATCATGTAACTCGCCTTGATCGTTGGGAACCGGAGCTGAATGAAGCCATACCAAA
CGACGAGCGTGACACCACGATGCCTGTAGCAATGGCAACAACGTTGCGCAAACTATTAACTGGCG
AACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGT
GGAAGCCGCGGTATCATTGCAGCACTGGGGCCAGATGGTAAGCCCTCCCGTATCGTAGTTATCTAC
ACGACGGGGAGTCAGGCAACTATGGATGAACGAAATAGACAGATCGCTGAGATAGGTGCCTCACT
GATTAAGCATTGGTAACTGTCAGACCAAGTTTACTCATATATACTTTAGATTGATTTAAAACTTCAT
TTTTAATTTAAAAGGATCTAGGTGAAGATCCTTTTTGATAATCTCATGACCAAAATCCCTTAACGTG
AGTTTTCGTTCCACTGAGCGTCAGACCCCGTAGAAAAGATCAAAGGATCTTCTTGAGATCCTTTTTT
TCTGCGCGTAATCTGCTGCTTGCAAACAAAAAAACCACCGCTACCAGCGGTGGTTTGTTTGCCGGA
TCAAGAGCTACCAACTCTTTTTCCGAAGGTAACTGGCTTCAGCAGAGCGCAGATACCAAATACTGT
TCTTCTAGTGTAGCCGTAGTTAGGCCACCACTTCAAGAACTCTGTAGCACCGCCTACATACCTCGC
TCTGCTAATCCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCACACAGCCCA
GCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGCCACCTCTGAC
TTGAGCGTCGATTTTTGTGATGCTCGTCAGGGGGGCGGAGCCTATGGAAAAACGCCAGCAACGCG
GCCTTTTTACGGTTCCTGGCCTTTTGCTGGCCTTTTGCTCACATGT
SEQ ID NO: 21-pAAV9-U6>RNA6-Mecp2 promoter>SadCas9/2xKRAB: SV40 pA
[sgRNA underlined, RNA6 (SEQ ID NO: 3); RNA6/sa optimized scaffold italicized]
CCTGCAGGCAGCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCATCAC
TAGGGGTTCCTTCTAGAGAGGGCCTATTTCCCATGATTCCTTCATATTTGCATATACGATACAAGGC
TGTTAGAGAGATAATTGGAATTAATTTGACTGTAAACACAAAGATATTAGTACAAAATACGTGAC
GTAGAAAGTAATAATTTCTTGGGTAGTTTGCAGTTTTAAAATTATGTTTTAAAATGGACTATCATAT
GCTTACCGTAACTTGAAAGTATTTCGATTTCTTGGCTTTATATATCTTGTGGAAAGGACGAAACAC
CGTGGCCATTCGACGACAGGTTAGTTATAGTACTCTGGAAACAGAATCTACTATAACAAGGCAAAATGCCG
TGTTTATCTCGTCAACTTGTTGGCGAGATTTTTTTAGCTGAATGGGGTCCGCCTCTTTTCCCTGCCTAAA
CAGACAGGAACTCCTGCCAATTGAGGGCGTCACCGCTAAGGCTCCGCCCCAGCCTGGGCTCCACA
ACCAATGAAGGGTAATCTCGACAAAGAGCAAGGGGTGGGGCGCGGGCGCGCAGGTGCAGCAGCA
CACAGGCTGGTCGGGAGGGGGGGGCGCGACGTCTGCCGTGCGGGGTCCCGGCATCGGTTGCGCGC
GCCACCATGTACCCATACGATGTTCCAGATTACGCTGCCCCAAAGAAGAAGCGGAAGGTCGGTAT
CCACGGAGTCCCAGCAGCCAAGCGGAACTACATCCTGGGCCTGGCCATCGGCATCACCAGCGTGG
GCTACGGCATCATCGACTACGAGACACGGGACGTGATCGATGCCGGCGTGCGGCTGTTCAAAGAG
GCCAACGTGGAAAACAACGAGGGCAGGCGGAGCAAGAGAGGCGCCAGAAGGCTGAAGCGGCGG
AGGCGGCATAGAATCCAGAGAGTGAAGAAGCTGCTGTTCGACTACAACCTGCTGACCGACCACAG
CGAGCTGAGCGGCATCAACCCCTACGAGGCCAGAGTGAAGGGCCTGAGCCAGAAGCTGAGCGAG
GAAGAGTTCTCTGCCGCCCTGCTGCACCTGGCCAAGAGAAGAGGCGTGCACAACGTGAACGAGGT
GGAAGAGGACACCGGCAACGAGCTGTCCACCAAAGAGCAGATCAGCCGGAACAGCAAGGCCCTG
GAAGAGAAATACGTGGCCGAACTGCAGCTGGAACGGCTGAAGAAAGACGGCGAAGTGCGGGGCA
GCATCAACAGATTCAAGACCAGCGACTACGTGAAAGAAGCCAAACAGCTGCTGAAGGTGCAGAA
GGCCTACCACCAGCTGGACCAGAGCTTCATCGACACCTACATCGACCTGCTGGAAACCCGGCGGA
CCTACTATGAGGGACCTGGCGAGGGCAGCCCCTTCGGCTGGAAGGACATCAAAGAATGGTACGAG
ATGCTGATGGGCCACTGCACCTACTTCCCCGAGGAACTGCGGAGCGTGAAGTACGCCTACAACGC
CGACCTGTACAACGCCCTGAACGACCTGAACAATCTCGTGATCACCAGGGACGAGAACGAGAAGC
TGGAATATTACGAGAAGTTCCAGATCATCGAGAACGTGTTCAAGCAGAAGAAGAAGCCCACCCTG
AAGCAGATCGCCAAAGAAATCCTCGTGAACGAAGAGGATATTAAGGGCTACAGAGTGACCAGCA
CCGGCAAGCCCGAGTTCACCAACCTGAAGGTGTACCACGACATCAAGGACATTACCGCCCGGAAA
GAGATTATTGAGAACGCCGAGCTGCTGGATCAGATTGCCAAGATCCTGACCATCTACCAGAGCAG
CGAGGACATCCAGGAAGAACTGACCAATCTGAACTCCGAGCTGACCCAGGAAGAGATCGAGCAG
ATCTCTAATCTGAAGGGCTATACCGGCACCCACAACCTGAGCCTGAAGGCCATCAACCTGATCCTG
GACGAGCTGTGGCACACCAACGACAACCAGATCGCTATCTTCAACCGGCTGAAGCTGGTGCCCAA
GAAGGTGGACCTGTCCCAGCAGAAAGAGATCCCCACCACCCTGGTGGACGACTTCATCCTGAGCC
CCGTCGTGAAGAGAAGCTTCATCCAGAGCATCAAAGTGATCAACGCCATCATCAAGAAGTACGGC
CTGCCCAACGACATCATTATCGAGCTGGCCCGCGAGAAGAACTCCAAGGACGCCCAGAAAATGAT
CAACGAGATGCAGAAGCGGAACCGGCAGACCAACGAGCGGATCGAGGAAATCATCCGGACCACC
GGCAAAGAGAACGCCAAGTACCTGATCGAGAAGATCAAGCTGCACGACATGCAGGAAGGCAAGT
GCCTGTACAGCCTGGAAGCCATCCCTCTGGAAGATCTGCTGAACAACCCCTTCAACTATGAGGTGG
ACCACATCATCCCCAGAAGCGTGTCCTTCGACAACAGCTTCAACAACAAGGTGCTCGTGAAGCAG
GAAGAAGCCAGCAAGAAGGGCAACCGGACCCCATTCCAGTACCTGAGCAGCAGCGACAGCAAGA
TCAGCTACGAAACCTTCAAGAAGCACATCCTGAATCTGGCCAAGGGCAAGGGCAGAATCAGCAAG
ACCAAGAAAGAGTATCTGCTGGAAGAACGGGACATCAACAGGTTCTCCGTGCAGAAAGACTTCAT
CAACCGGAACCTGGTGGATACCAGATACGCCACCAGAGGCCTGATGAACCTGCTGCGGAGCTACT
TCAGAGTGAACAACCTGGACGTGAAAGTGAAGTCCATCAATGGCGGCTTCACCAGCTTTCTGCGG
CGGAAGTGGAAGTTTAAGAAAGAGCGGAACAAGGGGTACAAGCACCACGCCGAGGACGCCCTGA
TCATTGCCAACGCCGATTTCATCTTCAAAGAGTGGAAGAAACTGGACAAGGCCAAAAAAGTGATG
GAAAACCAGATGTTCGAGGAAAAGCAGGCCGAGAGCATGCCCGAGATCGAAACCGAGCAGGAGT
ACAAAGAGATCTTCATCACCCCCCACCAGATCAAGCACATTAAGGACTTCAAGGACTACAAGTAC
AGCCACCGGGTGGACAAGAAGCCTAATAGAGAGCTGATTAACGACACCCTGTACTCCACCCGGAA
GGACGACAAGGGCAACACCCTGATCGTGAACAATCTGAACGGCCTGTACGACAAGGACAATGACA
AGCTGAAAAAGCTGATCAACAAGAGCCCCGAAAAGCTGCTGATGTACCACCACGACCCCCAGACC
TACCAGAAACTGAAGCTGATTATGGAACAGTACGGCGACGAGAAGAATCCCCTGTACAAGTACTA
CGAGGAAACCGGGAACTACCTGACCAAGTACTCCAAAAAGGACAACGGCCCCGTGATCAAGAAG
ATTAAGTATTACGGCAACAAACTGAACGCCCATCTGGACATCACCGACGACTACCCCAACAGCAG
AAACAAGGTCGTGAAGCTGTCCCTGAAGCCCTACAGATTCGACGTGTACCTGGACAATGGCGTGT
ACAAGTTCGTGACCGTGAAGAATCTGGATGTGATCAAAAAAGAAAACTACTACGAAGTGAATAGC
AAGTGCTATGAGGAAGCTAAGAAGCTGAAGAAGATCAGCAACCAGGCCGAGTTTATCGCCTCCTT
CTACAACAACGATCTGATCAAGATCAACGGCGAGCTGTATAGAGTGATCGGCGTGAACAACGACC
TGCTGAACCGGATCGAAGTGAACATGATCGACATCACCTACCGCGAGTACCTGGAAAACATGAAC
GACAAGAGGCCCCCCAGGATCATTAAGACAATCGCCTCCAAGACCCAGAGCATTAAGAAGTACAG
CACAGACATTCTGGGCAACCTGTATGAAGTGAAATCTAAGAAGCACCCTCAGATCATCAAAAAGG
GCAAAAGGCCGGCGGCCACGAAAAAGGCCGGCCAGGCAAAAAAGAAAAAGCATATGACTAGTGG
CGGTGGCGGAGGGATGGATGCTAAGTCACTAACTGCCTGGTCCCGGACACTGGTGACCTTCAAGG
ATGTATTTGTGGACTTCACCAGGGAGGAGTGGAAGCTGCTGGACACTGCTCAGCAGATCGTGTAC
AGAAATGTGATGCTGGAGAACTATAAGAACCTGGTTTCCTTGGGTTATCAGCTTACTAAGCCAGAT
GTGATCCTCCGGTTGGAGAAGGGAGAAGAGCCCACTAGTGGCGGTGGCGGAGGGATGGATGCTAA
GTCACTAACTGCCTGGTCCCGGACACTGGTGACCTTCAAGGATGTATTTGTGGACTTCACCAGGGA
GGAGTGGAAGCTGCTGGACACTGCTCAGCAGATCGTGTACAGAAATGTGATGCTGGAGAACTATA
AGAACCTGGTTTCCTTGGGTTATCAGCTTACTAAGCCAGATGTGATCCTCCGGTTGGAGAAGGGAG
AAGAGCCCTAGCTAGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGATGTGG
GAGGTTTTTTAAAGCATGCTGGGGAGGGCCGCAGGAACCCCTAGTGATGGAGTTGGCCACTCCCTC
TCTGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGGGGCGCCTGATGCGGTATTTTCTCCT
TACGCATCTGTGCGGTATTTCACACCGCATACGTCAAAGCAACCATAGTACGCGCCCTGTAGCGGC
GCATTAAGCGCGGCGGGGGTGGTGGTTACGCGCAGCGTGACCGCTACACTTGCCAGCGCCTTAGC
GCCCGCTCCTTTCGCTTTCTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGACCCCAAAAAACTTGATT
TGGGTGATGGTTCACGTAGTGGGCCATCGCCCTGATAGACGGTTTTTCGCCCTTTGACGTTGGAGT
CCACGTTCTTTAATAGTGGACTCTTGTTCCAAACTGGAACAACACTCAACTCTATCTCGGGCTATTC
TTTTGATTTATAAGGGATTTTGCCGATTTCGGTCTATTGGTTAAAAAATGAGCTGATTTAACAAAA
ATTTAACGCGAATTTTAACAAAATATTAACGTTTACAATTTTATGGTGCACTCTCAGTACAATCTGC
TCTGATGCCGCATAGTTAAGCCAGCCCCGACACCCGCCAACACCCGCTGACGCGCCCTGACGGGCT
TGTCTGCTCCCGGCATCCGCTTACAGACAAGCTGTGACCGTCTCCGGGAGCTGCATGTGTCAGAGG
TTTTCACCGTCATCACCGAAACGCGCGAGACGAAAGGGCCTCGTGATACGCCTATTTTTATAGGTT
AATGTCATGATAATAATGGTTTCTTAGACGTCAGGTGGCACTTTTCGGGGAAATGTGCGCGGAACC
CCTATTTGTTTATTTTTCTAAATACATTCAAATATGTATCCGCTCATGAGACAATAACCCTGATAAA
TGCTTCAATAATATTGAAAAAGGAAGAGTATGAGTATTCAACATTTCCGTGTCGCCCTTATTCCCTT
TTTTGCGGCATTTTGCCTTCCTGTTTTTGCTCACCCAGAAACGCTGGTGAAAGTAAAAGATGCTGA
AGATCAGTTGGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGAATGACTTGGT
TGAGTACTCACCAGTCACAGAAAAGCATCTTACGGATGGCATGACAGTAAGAGAATTATGCAGTG
CTGCCATAACCATGAGTGATAACACTGCGGCCAACTTACTTCTGACAACGATCGGAGGACCGAAG
GAGCTAACCGCTTTTTTGCACAACATGGGGGATCATGTAACTCGCCTTGATCGTTGGGAACCGGAG
CTGAATGAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACTGGATGGA
GGCGGATAAAGTTGCAGGACCACTTCTGCGCTCGGCCCTTCCGGCTGGCTGGTTTATTGCTGATAA
ATCTGGAGCCGGTGAGCGTGGAAGCCGCGGTATCATTGCAGCACTGGGGCCAGATGGTAAGCCCT
CCCGTATCGTAGTTATCTACACGACGGGGAGTCAGGCAACTATGGATGAACGAAATAGACAGATC
GCTGAGATAGGTGCCTCACTGATTAAGCATTGGTAACTGTCAGACCAAGTTTACTCATATATACTT
TAGATTGATTTAAAACTTCATTTTTAATTTAAAAGGATCTAGGTGAAGATCCTTTTTGATAATCTCA
TGACCAAAATCCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAACCACCGCTACC
AGCGGTGGTTTGTTTGCCGGATCAAGAGCTACCAACTCTTTTTCCGAAGGTAACTGGCTTCAGCAG
AGCGCAGATACCAAATACTGTTCTTCTAGTGTAGCCGTAGTTAGGCCACCACTTCAAGAACTCTGT
AGCACCGCCTACATACCTCGCTCTGCTAATCCTGTTACCAGTGGCTGCTGCCAGTGGCGATAAGTC
GTGTCTTACCGGGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGATACCTACAGCGT
GAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTTTTGCTCACATGT
SEQ ID NO: 22-RNA6/sa optimized scaffold [sgRNA underlined, RNA6 (SEQ ID NO: 3)]
GTGGCCATTCGACGACAGGTTAGTTATAGTACTCTGGAAACAGAATCTACTATAA
CAAGGCAAAATGCCGTGTTTATCTCGTCAACTTGTTGGCGAGATTTTTTT
SEQ ID NO: 23-RNA6/sa optimized scaffold complement (3'->5') [sgRNA underlined,
complement of RNA6 (SEQ ID NO: 3)]
CACCGGTAAGCTGCTGTCCAATCAATATCATGAGACCTTTGTCTTAGATGATATT
GTTCCGTTTTACGGCACAAATAGAGCAGTTGAACAACCGCTCTAAAAAAA
SEQ ID NO: 24-HEK-155R (TSS2-sg1)
GAATGGTCGTGGGCACCGGGA
SEQ ID NO: 25-HEK-571F (TSS2-sg3)
ACGCTCTCGGAGGGGCCGGGC
SEQ ID NO: 26-HEK-469F (TSS2-sg4)
GGCAAACCCGCTAACCTGTCG
SEQ ID NO: 27-HEK-267R (TSS2-sg5)
AAGAGAGAGGCGGGGAGGAGT
SEQ ID NO: 28-HEK-453R (TSS2-sg2, an alternative embodiment to SEQ ID NO: 3);
TGGCCATTCGACGACAGGTTA
SEQ ID NO: 29-addgene-plasmid-163022-sequence-320905 (683..4120) (3438 bp;
containing a mammalian codon-optimized SadCas9)
cggacactagtgaccttcaaggatgtatttgtggacttcaccagggaggagtggaagctgctggacactgctcagcagat
cgtgtacagaaatgtgatgctggagaactataagaacctggtttccttgggttatcagcttactaagccagatgtgatcc
tccggttggagaagggagaagagcccggggttccggcggagggtccatgggcaaacggaactacatcctggggcttgcc
attgggataaccagcgttggctacggaattattgattatgagacacgcgatgtgattgacgccggggttaggctgttcaa
agaggccaacgttgaaaacaacgagggaagacggagtaagcgcggagcaagaagactcaagcgcagacggagacatcgga
ttcagagggtgaaaaagctgctcttcgattacaatctcctgaccgatcatagtgagctgagcggaatcaacccctacgag
gcgcgagtgaaagggctttcccagaagctgtccgaagaggagttctccgccgcgttgctgcacctggccaaacggagggg
ggttcacaatgtaaacgaagtggaggaggacacgggcaatgaacttagtacgaaagaacagatcagtaggaactctaagg
ctctcgaagagaaatacgtcgctgagttgcagcttgagagactgaaaaaagacggcgaagtacgcggatctattaatagg
ttcaagacttcagattacgtaaaggaagccaagcagctcctgaaagtacagaaagcgtaccatcagctcgatcagagctt
catgatacctacatagatttgctggagacacggaggacatactacgagggcccaggggaaggatctccttttgggtgga
aggacatcaaggaatggtacgagatgcttatgggacattgtacatattttccggaggagctcaggagcgtcaagtacgcc
tacaatgccgacctgtacaatgccctcaatgacctcaataacctcgtgattaccagggacgagaacgagaagctggagta
ctatgaaaagttccagattatcgagaatgtgtttaagcagaagaagaagccgacacttaagcagattgcaaaggaaatcc
tcgtgaatgaggaagatatcaagggatacagagtgacaagtacaggcaagcccgagttcacaaatctgaaggtgtaccac
gatattaaggacataaccgcacgaaaggagataatcgaaaacgctgagctcctcgatcagatcgcaaaaattcttaccat
ctaccagtctagtgaggacattcaggaggaactgactaatctgaacagtgagctcacccaagaggaaattgagcagattt
caaacctgaaaggctacaccgggacgcacaatctgagcctcaaagcaatcaacctcattctggatgaactttggcacaca
aatgacaaccaaattgccatattcaaccgcctgaaactggtgccaaaaaaagtggatctgtcacagcaaaaggaaatccc
tacaaccttggttgacgattttattctgtcccccgttgtcaagcggagcttcatccagtcaatcaaggtgatcaatgcca
tcattaaaaaatacggattgccaaacgatataattatcgagcttgcacgagagaagaactcaaaggacgcccagaagatg
attaacgaaatgcagaagcgcaaccgccagacaaacgaacgcatagaggaaattataagaacaaccggcaaagagaatgc
caagtatctgatcgagaaaatcaagctgcacgacatgcaagaaggcaagtgcctgtactctctggaagctatcccactcg
aagatctgctgaataatccattcaattacgaggtggacgccatcatccctagatccgtaagctttgacaattccttcaat
aacaaagttctggttaaacaggaggaaaattctaaaaaagggaaccggaccccgttccagtacctgagctccagtgacag
caagattagctacgagacttttaagaaacatattctgaatctggccaaaggcaaaggcaggatcagcaagaccaagaagg
agtacctcctcgaagaacgcgacattaacagatttagtgtgcagaaagatttcatcaaccgaaaccttgtcgatactcgg
tacgccacgagaggcctgatgaatctcctcaggagctacttccgcgtcaataatctggacgttaaagtcaagagcataaa
tgggggattcaccagctttctgaggagaaagtggaagtttaagaaggaacgaaacaaaggatacaagcaccatgctgagg
atgctttgatcatcgctaacgcggactttatctttaaggaatggaaaaagctggataaggcaaagaaagtgatggaaaac
cagatgttcgaggagaagcaggcagagtcaatgcctgagatcgagacagagcaggaatacaaggaaattttcatcacccc
tcatcagattaaacacataaaggacttcaaagactataaatactctcatagggtggacaaaaaacccaatcgcgagctca
ttaatgacaccctgtactcaacacggaaggatgataaaggtaataccttgattgtgaataatcttaatggattgtatgac
aaagataacgacaagctcaagaagctgatcaacaagtctccagagaagctccttatgtatcaccacgacccacagactta
tcagaaattgaaactgatcatggagcaatacggggatgagaagaacccactctacaaatattatgaggaaacaggtaatt
acctgaccaagtactccaagaaggataacggaccagtgatcaaaaagataaagtactatggcaacaaacttaatgcgcat
ttggacataactgacgattaccccaattctcgaaacaaggttgtgaagctctccctgaagccttatagatttgacgtgta
cctggataatggggtttataaattcgtcaccgtgaaaaatctggacgtgatcaaaaaggagaactattatgaagtaaact
caaagtgctatgaggaggcgaagaagctgaagaagatctccaatcaggccgagttcatcgcttccttctataataacgat
ctcatcaagatcaatggagagctttatcgcgtcattggtgtgaacaatgacttgctgaacaggatcgaagtcaatatgat
agacattacctaccgggagtatctcgaaaacatgaatgataaacggccgcctcgcatcatcaagacaatcgcatctaaaa
ctcagtcaataaaaaagtactctaccgatatcctggggaatctctatgaagtgaagtcaaagaagcacccacaaatcatt
aaaaaaggtggatccgcagaagcagcagcaaaggaagcagcagcaaaggaagcagcagcaaaggcagtcgacaccggt
SEQ ID NO: 30-addgene-plasmid-135338-sequence-274405 (1152..4400) (3249 bp)
ccaaagaagaagcggaaggtcggtatccacggagtcccagcagccaagcggaactacatcctgggcctggccatcggcat
caccagcgtgggctacggcatcatcgactacgagacacgggacgtgatcgatgccggcgtgcggctgttcaaagaggcca
acgtggaaaacaacgagggcaggggagcaagagaggcgccagaaggctgaagcggcggaggggcatagaatccagaga
gtgaagaagctgctgttcgactacaacctgctgaccgaccacagcgagctgagcggcatcaacccctacgaggccagagt
gaagggcctgagccagaagctgagcgaggaagagttctctgccgccctgctgcacctggccaagagaagaggcgtgcaca
acgtgaacgaggtggaagaggacaccggcaacgagctgtccaccaaagagcagatcagccggaacagcaaggccctggaa
gagaaatacgtggccgaactgcagctggaacggctgaagaaagacggcgaagtgcggggcagcatcaacagattcaagac
cagcgactacgtgaaagaagccaaacagctgctgaaggtgcagaaggcctaccaccagctggaccagagcttcatcgaca
cctacatcgacctgctggaaacccggcggacctactatgagggacctggcgagggcagccccttcggctggaaggacatc
aaagaatggtacgagatgctgatgggccactgcacctacttccccgaggaactgcggagcgtgaagtacgcctacaacgc
cgacctgtacaacgccctgaacgacctgaacaatctcgtgatcaccagggacgagaacgagaagctggaatattacgaga
agttccagatcatcgagaacgtgttcaagcagaagaagaagcccaccctgaagcagatcgccaaagaaatcctcgtgaac
gaagaggatattaagggctacagagtgaccagcaccggcaagcccgagttcaccaacctgaaggtgtaccacgacatcaa
ggacattaccgcccggaaagagattattgagaacgccgagctgctggatcagattgccaagatcctgaccatctaccaga
gcagcgaggacatccaggaagaactgaccaatctgaactccgagctgacccaggaagagatcgagcagatctctaatctg
aagggctataccggcacccacaacctgagcctgaaggccatcaacctgatcctggacgagctgtggcacaccaacgacaa
ccagatcgctatcttcaaccggctgaagctggtgcccaagaaggtggacctgtcccagcagaaagagatccccaccaccc
tggtggacgacttcatcctgagccccgtcgtgaagagaagcttcatccagagcatcaaagtgatcaacgccatcatcaag
aagtacggcctgcccaacgacatcattatcgagctggcccgcgagaagaactccaaggacgcccagaaaatgatcaacga
gatgcagaagcggaaccggcagaccaacgagcggatcgaggaaatcatccggaccaccggcaaagagaacgccaagtacc
tgatcgagaagatcaagctgcacgacatgcaggaaggcaagtgcctgtacagcctggaagccatccctctggaagatctg
ctgaacaaccccttcaactatgaggtggaccacatcatccccagaagcgtgtccttcgacaacagcttcaacaacaaggt
gctcgtgaagcaggaagaagccagcaagaagggcaaccggaccccattccagtacctgagcagcagcgacagcaagatca
gctacgaaaccttcaagaagcacatcctgaatctggccaagggcaagggcagaatcagcaagaccaagaaagagtatctg
ctggaagaacgggacatcaacaggttctccgtgcagaaagacttcatcaaccggaacctggtggataccagatacgccac
cagaggcctgatgaacctgctgcggagctacttcagagtgaacaacctggacgtgaaagtgaagtccatcaatggcggct
tcaccagctttctgcggcggaagtggaagtttaagaaagagcggaacaaggggtacaagcaccagccgaggacgccctg
atcattgccaacgccgatttcatcttcaaagagtggaagaaactggacaaggccaaaaaagtgatggaaaaccagatgtt
cgaggaaaagcaggccgagagcatgcccgagatcgaaaccgagcaggagtacaaagagatcttcatcaccccccaccaga
tcaagcacattaaggacttcaaggactacaagtacagccaccgggtggacaagaagcctaatagagagctgattaacgac
accctgtactccacccggaaggacgacaagggcaacaccctgatcgtgaacaatctgaacggcctgtacgacaaggacaa
tgacaagctgaaaaagctgatcaacaagagccccgaaaagctgctgatgtaccaccacgacccccagacctaccagaaac
tgaagctgattatggaacagtacggcgacgagaagaatcccctgtacaagtactacgaggaaaccgggaactacctgacc
aagtactccaaaaaggacaacggccccgtgatcaagaagattaagtattacggcaacaaactgaacgcccatctggacat
caccgacgactaccccaacagcagaaacaaggtcgtgaagctgtccctgaagccctacagattcgacgtgtacctggaca
atggcgtgtacaagttcgtgaccgtgaagaatctggatgtgatcaaaaaagaaaactactacgaagtgaatagcaagtgc
tatgaggaagctaagaagctgaagaagatcagcaaccaggccgagtttatcgcctccttctacaacaacgatctgatcaa
gatcaacggcgagctgtatagagtgatcggcgtgaacaacgacctgctgaaccggatcgaagtgaacatgatcgacatca
cctaccgcgagtacctggaaaacatgaacgacaagaggccccccaggatcattaagacaatcgcctccaagacccagagc
attaagaagtacagcacagacattctgggcaacctgtatgaagtgaaatctaagaagcaccctcagatcatcaaaaaggg
caaaaggccggcggccacgaaaaaggccggccaggcaaaaaagaaaaag

What is claimed is:

1. A recombinant DNA molecule comprising a nucleic acid sequence that encodes a fusion protein comprising a clustered regularly interspaced short palindromic repeats (CRISPR)-associated nuclease and a transcriptional repressor.

2. The recombinant DNA molecule of claim 1, further comprising a nucleic acid sequence that encodes a guide RNA that is complementary to a sequence in an alpha-synuclein gene or a gene regulatory region thereof.

3. The recombinant DNA molecule of claim 2, wherein the alpha-synuclein gene is a human or non-human primate alpha-synuclein gene.

4. The recombinant DNA molecule of claim 2, wherein the nucleic acid sequence that encodes a guide RNA comprises a sequence that is at least 90% identical to SEQ ID NO: 1, 2, 3, 24, 25, 26, 27, or 28.

5. The recombinant DNA molecule of claim 1, wherein the CRISPR-associated nuclease is Cas9.

6. The recombinant DNA molecule of claim 5, wherein the Cas9 is Staphylococcus aureus Cas9.

7. The recombinant DNA molecule of claim 1, wherein the CRISPR-associated nuclease comprises one or more mutations relative to the wild-type CRISPR-associated nuclease.

8. The recombinant DNA molecule of claim 7, wherein at least one of the one or more mutations results in a reduction in CRISPR-associated nuclease catalytic activity.

9. The recombinant DNA molecule of claim 8, wherein the one or more mutations results in a catalytically inactive CRISPR-associated nuclease.

10. The recombinant DNA molecule of claim 7, wherein the CRISPR-associated nuclease is S. aureus Cas9 and wherein the S. aureus Cas9 comprises a D10A mutation and/or a N580A mutation.

11. The recombinant DNA molecule of claim 1, wherein the transcriptional repressor is a Krüppel associated box (KRAB) domain.

12. The recombinant DNA molecule of claim 11, wherein the fusion protein comprises at least 2 KRAB domains.

13. The recombinant DNA molecule of claim 1, wherein the fusion protein further comprises a nuclear localization signal (NLS).

14. A DNA construct comprising the recombinant DNA molecule of claim 1 operably linked to a constitutive or inducible promoter.

15. The DNA construct of claim 14, wherein the promoter is a human methyl CpG binding protein 2 (MECP2) promoter, a human synapsin promoter (SYN1), or a human phosphoglycerate kinase (PGK) promoter.

16. The DNA construct of claim 14, wherein the DNA construct comprises two promoters: i) a first promoter operably linked to the nucleic acid sequence that encodes a fusion protein comprising a CRISPR-associated nuclease and a transcriptional repressor; and ii) a second promoter operably linked to the nucleic acid sequence that encodes a guide RNA that is complementary to a sequence in an alpha-synuclein gene or a gene regulatory region thereof.

17. The DNA construct of claim 16, wherein the first promoter is a human MECP2 promoter, a human synapsin promoter, or a human PGK promoter.

18. The DNA construct of claim 16, wherein the second promoter is a human U6 promoter.

19. A vector comprising the DNA construct of claim 14.

20. The vector of claim 19, wherein the vector is a viral vector.

21. The vector of claim 20, wherein the vector is an adeno-associated virus vector.

22. The vector of claim 21, wherein the adeno-associated virus is AAV9.

23. The vector of claim 19, wherein the vector is up to 4800 bp in length.

24. The vector of claim 23, wherein the vector is up to 5100 bp in length.

25. The vector of claim 19, wherein the DNA construct comprises a sequence that is at least 90% identical to SEQ ID NO:20 or 21.

26. An isolated virus comprising the vector of claim 19.

27. The isolated virus of claim 26, wherein the virus is an adeno-associated virus.

28. The isolated virus of claim 27, wherein the adeno-associated virus is AAV9.

29. A composition comprising:
i) the recombinant DNA molecule of claim 1, the DNA construct of claim 14, the vector of claim 19, or the isolated virus of claim 26; and
ii) a pharmaceutically acceptable carrier.

30. A method of treating a subject with Parkinson's disease, the method comprising administering to the subject a therapeutically effective amount of the composition of claim 29.

31. The method of claim 30, wherein the composition is administered intrathecally.

32. The method of claim 30, wherein the composition is administered into the cisterna magna.

33. The method of claim 30, wherein the composition is administered into cerebrospinal fluid.

34. The method of claim 30, wherein the administration of the composition results in a decreased amount of alpha-synuclein protein expression in the subject relative to the amount of alpha-synuclein protein expression in the subject prior to administration of the composition.
